(19) United States
(12) Patent Application Publication
(10) Pub. No.: US 2024/0093213 A1
(43) Pub. Date: Mar. 21, 2024

(54) PROCESSES, SYSTEMS AND MEDIA FOR DELIVERING A SUBSTANCE TO A PLANT

(71) Applicant: EPIGENETICA LIMITED, Glasgow, Strathclyde (GB)

(72) Inventor: Jennifer STEPHENS, Dundee (GB)

(73) Assignee: EPIGENETICA LIMITED, Glasgow, Strathclyde (GB)

(21) Appl. No.: 18/255,086

(22) PCT Filed: Nov. 30, 2021
(86) PCT No.: PCT/EP2021/083668

§ 371 Date: May 30, 2023

(30) Foreign Application Priority Data
Dec. 1, 2020 (GB) 2018966.8

(51) Int. Cl. C12N 15/82

(52) U.S. Cl. CPC C12N 15/8206

(57) ABSTRACT

The invention relates to a process for delivering a substance, optionally a compound, vector or nanomaterial, to a plant. The process comprises providing a plant application medium comprising a substance, a carrier medium and micro- and/or nanobubbles of at least one gas; and applying the plant application medium to a locus of a plant. The substance enters at least one plant tissue of the plant. The substance may be one or more substances for inducing a change in a phenotype, chemistry or physiology of a plant, for example an epigenetic regulator. The present invention also relates to a system for delivering a substance to a plant and to media to be applied to a plant.

### FIELD OF INVENTION

The present invention relates to processes, systems and media for delivering a substance to a plant. More particularly, the present invention includes methods and associated systems for cultivating a plant including a step of providing nano- and/or microbubbles and one or more substances, for example providing nanobubbles and a substance at the root of a plant, whereby the substance is delivered to plant cells. The substance may, for example, be useful in altering physiology and/or gene expression.

### BACKGROUND

Plants produce a large number of molecules which may be utilised, for example as foods, drugs, colorants, flavourings, comestible additives or crop protection products (for example fungicides, nematicides, pesticides or the like). These molecules may not be essential to the survival of the plant and thus only expressed under particular conditions and/or only expressed at low levels. Chemical synthesis of such molecules by the plant may be the most efficient synthesis route to generate the molecule(s) for commercial use, for example where the molecules are complex and/or extraction from plants remain the best sources of supply.

Soil-less growth, for example hydroponic growth systems, which allow plant growth under controlled conditions in a greenhouse or outdoors have developed considerably over recent years. Although modulation of growth conditions to allow improved production of secondary metabolites from plants has been provided, further improvements are required.

EP 2 761 993 relates to a method for cultivating a plant using an artificial light-irradiating lamp wherein a plant is irradiated with a red light and then with a blue light, for a predetermined period of time, wherein the cultivation conditions include providing dissolved oxygen in a nutritious liquid.

WO 2017/156410 discusses providing a composition containing nanobubbles dispersed in a liquid carrier with another liquid to create an oxygen-enriched composition that is then applied to plant roots. Such a composition can promote germination or growth of plant seedlings.

EP 2 460 582 discusses the production of super-micro bubbles of several hundred nm to several dozen μm in size (diameter) and ways in which such bubbles can be provided.

EP 3 721 979 relates to a charged nanobubble dispersion liquid, a manufacturing method thereof and manufacturing apparatus therefor, and a method to control the growth rate of microorganisms and plants using nanobubble dispersion liquid.

US 2020/0045980 discusses the use of one or more volatile organic compounds produced by Cladosporium sphaerospermum to increase at least one growth characteristic in a plant after exposure of the plant to the volatile organic compound(s) (VOCs) wherein the VOCs from Cladosporium sphaerospermum were provided to the plant's headspace. Cladosporium sphaerospermum was noted not to be required to grow in the soil with the plant to be treated as; in fact, such growth in soil may result in reduced effects on the plant's phenotype (growth, yield, etc). Methods have been provided to provide VOCs into plant cells (Li Zhijian T., Janisiewicz Wojciech J., Liu Zongrang, Callahan Ann M., Evans Breyn E., Jurick Wayne M., Dardick Chris. (2019). Exposure in vitro to an Environmentally Isolated Strain TC09 of Cladosporium sphaerospermum Triggers Plant Growth Promotion, Early Flowering, and Fruit Yield Increase. Frontiers in Plant Science, 9 1959; but alternative introduction methods are required.

Various methods have been used to introduce short fragments of DNA (antisense oligonucleotides) or small RNAs into plant cells with only limited success.

### SUMMARY OF INVENTION

Without proper oxygenation, plants growing in hydroponic solutions die. The application of oxygen to the water in the form of nano- or microbubbles maintains a level of dissolved oxygen in the water that enables roots to absorb nutrients for growth. The use of nanobubbles in the growth of plants to date has been to provide oxygen to promote growth or as an additive to standard growth fertiliser compositions.

The present inventors have determined that nano- and/or microbubbles provided in combination with a compound or substance, wherein the compound or substance is attached to the bubble, in the bubble or in solution with the bubble, allows transport of the compound or substance within a plant/plant cells. The combination of nano- and/or microbubbles and compounds or substances in or attached to such a bubble, or in solution with these nano- and/or microbubbles can be used to alter, for example, gene expression. It is considered this provides an advantageous way to transport exogenous compounds or substances to cells in the plant.

In particular, it is considered the present technology enables control of plant gene expression during growth, in real-time and in commercial environments. This enables crop production with higher yields, production of new compounds by plants, production of increased yields of compounds in plants, and features like ‘flowering on demand’. For example, production of compounds in the plant may be through the manipulation of latent and active biosynthetic pathways in the plant.

In a first aspect, the present invention provides a plant cultivation system comprising: (i) a micro- and/or nanobubble generating apparatus for generating micro- and/or nanobubbles from at least one gas; (ii) a plant application medium comprising a substance, a carrier medium and micro- and/or nanobubbles formed from at least one gas by the micro- and/or nanobubble generating apparatus; and (iii) an applicator system to apply the plant application medium to a locus of a plant.

Advantageously, the substance is at least one substance capable of inducing a change in the phenotype, genotype, chemistry, or physiology of the plant

In one embodiment, the applicator system comprises a system for immersion of roots and/or leaves of the plant in the plant application medium.

In certain examples, the applicator system comprises a system for spraying, fogging or misting the plant with the plant application medium.

Optionally, the at least one gas comprises carbon dioxide and the applicator system comprises a system for misting leaves of the plant.

In certain embodiments, the applicator system is in fluid communication with the micro- and/or nanobubble generating apparatus.

In some embodiments, the system comprises a hydroponic plant cultivation system.

In certain preferred embodiments, the micro- and/or nanobubble generating apparatus is a nanobubble-generating apparatus.

In some examples, the substance is at least one compound, vector or nanomaterial.

Optionally, the substance comprises an epigenetic regulator.

In further examples, the substance is at least one substance selected from: volatile organic compounds (VOCs); transgenes, nucleic acids, DNAs, RNAs, siRNA, antisense oligonucleotides, synthetic or native DNA or RNA, synthetic or native DNA or RNA up to 500 nucleotides, optionally up to 200 nucleotides; plant growth regulators, gibberellins, auxins, abscisic acid, cytokinins and ethylene; epigenetic regulators; RNAi vectors, expression vectors, viral vectors, mono-polysaccharides; polyphenols; terpenoids; proteins or peptides, optionally peptides up to 150 amino acids, optionally up to 50 amino acids; nanomaterials, optionally a nanomaterial selected from: lipid nanoparticles, carbon nanotubes, copper nanoparticles, iron or iron oxide nanoparticles, manganese or manganese oxide nanoparticles, titanium dioxide nanoparticles, and zinc or zinc oxide nanoparticles; and plant protection products.

In some preferred examples, the substance is at least one substance selected from VOCs, RNAs, siRNA, antisense oligonucleotides, epigenetic regulators, peptides, RNAi vectors, expression vectors and viral vectors. There groups are not mutually exclusive. In other words, the substance may belong to more than one of these groups.

Optionally, the substance is a nanomaterial selected from: lipid nanoparticles, carbon nanotubes, copper nanoparticles, iron or iron oxide nanoparticles, manganese or manganese oxide nanoparticles, titanium dioxide nanoparticles, and zinc or zinc oxide nanoparticles.

The application of phyto-nanotechnology is reviewed in Environ. Sci.: Nano, 2020, 7, 2863-2874, to which further reference should be made.

In a second aspect, the present invention provides a process for delivering a substance to cells of a plant, the process comprising: (i) providing a plant application medium comprising a substance, a carrier medium and micro- and/or nanobubbles of at least one gas; and (ii) applying the plant application medium to a locus of a plant.

Suitably, the substance is a substance as defined above with respect to the first aspect of the present invention.

Optionally, the step of applying the plant application medium to the plant comprises applying the plant application medium to roots and/or leaves of the plant, optionally by immersion, spraying, fogging or misting.

Advantageously, the substance and micro- and/or nanobubbles are transported or translocated from the locus of the plant to at least one plant cell, optionally wherein the substance and micro- and/or nanobubbles are transported or translocated from a first plant tissue to a second plant tissue.

In a third aspect, the present invention provides a plant application medium for applying to a locus of plant, the medium comprising a substance, a carrier medium and micro- and/or nanobubbles of at least one gas.

In a fourth aspect, the present invention also provides a plant to which the medium has been applied to a locus thereof.

Suitably, the substance of the third and fourth aspects is a substance as defined above with respect to the first aspect of the present invention.

Suitably, the locus is roots of the plant or leaves of the plant.

In a fifth aspect, the present invention provides a process for inducing a change in a phenotype, chemistry or physiology of a plant by delivering an epigenetic regulator to a plant, the process comprising: (i) providing a plant application medium comprising a substance, a carrier medium and micro- and/or nanobubbles of at least one gas; and (ii) applying the plant application medium to a plant, whereby the epigenetic regulator enters at least one plant tissue of the plant and a subsequent change is induced in the phenotype, chemistry or physiology of the plant.

Advantageously, the epigenetic regulator is selected from: volatile organic compound(s) (VOC(s)), optionally fungal, microbial or plant VOCs; RNA, siRNA; antisense oligonucleotides; peptides; viral vectors; and plant growth regulators.

In some examples, in use of the process, the epigenetic regulator induces DNA methylation, RNA methylation, histone methylation or histone acetylation, optionally in one or more flowering loci.

In some examples, the plant epigenetic regulator is a nucleic acid.

In other examples, the epigenetic regulator is at least one RNAi vector and/or expression vector.

In a sixth aspect, the present invention provides a process for editing a gene of a plant, the process comprising: (i) providing a plant application medium comprising a gene editing substance, a carrier medium and micro- and/or nanobubbles of at least one gas; and (ii) applying the plant application medium to a plant, whereby the substance enters at least one plant cell.

Advantageously, the substance comprises a CRISPR/Cas9 construct, optionally wherein the substance comprises a CRISPR/Cas9 construct introduced via Agrobacterium.

In certain examples, the substance contains vectors expressing the 13 glucosidase gene.

In a seventh aspect, the present invention provides a process for delivering a plant or crop protection product into a plant, the process comprising: (i) providing a plant application medium comprising a substance, a carrier medium and micro- and/or nanobubbles of at least one gas; and (ii) applying the plant application medium to a plant; wherein the substance is at least one plant or crop protection product.

Optionally, the plant or crop protection product is a herbicide or pesticide, optionally an insecticide, nematocide or acaricide.

In use of the process, the plant or crop protection product is absorbed into a plant tissue, optionally a leaf or root tissue.

In an eighth aspect, the present invention provides a process for delivering an antisense oligonucleotide to a plant, the process comprising: (i) providing a plant application medium comprising a substance, a carrier medium and micro- and/or nanobubbles of at least one gas; and (ii) applying the plant application medium to a plant; wherein the substance is at least one antisense oligonucleotide.

In use of the process, the antisense oligonucleotide enters at least one plant cell of the plant.

Optionally, the antisense oligonucleotide plant application medium is applied to a root of the plant, further optionally wherein the antisense oligonucleotide is translocated from the root of the plant to a leaf of the plant, in use of the process.

Optionally, the antisense oligonucleotide is a labelled antisense oligonucleotide.

Optionally, in any aspect of the present invention, at least 50%, of the micro and/or nanobubbles generated have a diameter of less than about 1000 nm, optionally less than about 500 nm, optionally about 20 nm, optionally in a range from 10 nm to 150 nm, optionally 2 nm or less.

Further optionally, in any aspect of the present invention, 100%, or about 100%, of the micro- and/or nanobubbles generated have a diameter of less than about 1000 nm, optionally less than about 500 nm, optionally about 20 nm, optionally in a range from nm to 150 nm, optionally 2 nm or less

Optionally, in any aspect of the present invention, the at least one gas is selected from oxygen, nitrogen, carbon dioxide and air.

Optionally, in any aspect of the present invention, the nanobubbles are generated using an electric field.

Advantageously, in any aspect of the present invention, the nanobubbles generated maintain stability for about 2 years or longer.

In certain examples of the processes of the present invention, the process further comprises a pre-treatment step wherein rooted shoots of the plant are incubated in an oxygen nanobubble water for one to two days prior to application of the medium.

In certain examples of the processes of the present invention, a mixture of a nanobubble water and one or more substance to alter gene expression is provided to a plant at any time in the life cycle of the plant to induce one or more epigenetic changes in real time.

Optionally, in any aspect of the present invention, the plant is Cannabis sativa, Nicotiana benthamiana, Hordeum vulgare, Nicotiana tabacum, Lactuca sativa or Ocimum basilicum.

Suitably the nanobubbles and/or microbubbles may be generated in a liquid medium, for example a liquid growth medium, a sugar-containing solution or water.

Suitably the one or more substances capable of inducing a change in the phenotype, genotype, chemistry, or physiology of a plant is a specific compound, that can be used to specifically enhance a plant in a desired way.

Suitably the compound may alter growth, alter flowering (for example bring forward flowering/provide earlier reproduction), alter crop productivity, for example fruit production (for example, increase yields).

Suitably the compound may increase the amount of a primary or secondary metabolite provided by the plant.

Suitably the compounds may improve the uptake or availability of essential nutrients within the plant to allow for increased plant growth.

Suitably, the compound(s) may be capable of activating plant defenses and/or stimulating pathways which provide protection against biotic and abiotic stresses.

Suitably, the compound or substance may be within the micro- and/or nanobubble, attached to the micro- and/or nanobubble, or may be in solution with (not attached) to the micro- and/or nanobubble or combinations thereof.

It is considered that previous improvements in growth are limited to micro- and/or nanobubbles improving uptake of nutrients (mainly nitrogen, phosphorous or potassium) or basic fertilisers at the roots of the plants only. It is not considered there has been any previous teaching or description of micro-nanobubbles entering the plant and transporting compounds into the plant cells, in particular to plant cells in the leaf or aerial portions of the plant. In particular it is not considered there has been any previous discussions of micro- and/or nanobubbles enhancing the uptake of genetic material, for example nucleic acid, for example RNA, DNA, microRNA, RNAi, double stranded DNA or RNA fragments or the like and/or in increasing the transport of such genetic material within a plant following uptake (for example to the leaf, flowering portions or aerial portions of the plant).

Nitrogen, phosphorus and potassium are typically considered essential for the growth of plants. Other nutrients such as minerals including, for example, calcium, magnesium and iron may be provided in a growth liquid for the plant. Suitably nitrogen fertilizers such as ammonium sulphate, ammonium chloride, ammonium nitrate, urea, nitrogenous lime, potassium nitrate, calcium nitrate and sodium nitrate; phosphate fertilizers such as superphosphate of lime and fused magnesium phosphate; potassium fertilizers such as potassium chloride and potassium sulphate; and minerals such as calcium, magnesium and iron may be provided to a plant growth solution.

Suitably, nutrients essential for normal plant growth may not be encompassed by the term ‘compounds’ as used herein. Suitably, in the present invention such nitrogen, phosphorus and potassium or a mineral such as calcium, magnesium and iron may not be considered to be one or more compounds capable of inducing a change in the phenotype, chemistry, or physiology of a plant.

Suitably the mixture to be applied to the plant may further comprise one or more further constituents such as surfactants, chelating agents, emulsifiers, antioxidants, reductants, pH and osmotic buffers.

Suitably a nucleic acid construct, for example short interfering RNA (siRNA), antisense oligonucleotide, microRNA or the like may be selected to target a gene responsible for a pathway in a plant, for example a pathway responsible for production of a secondary metabolite in a plant. Any suitable secondary metabolite may be selected.

Suitably a secondary plant metabolite may include phenolics, alkaloids, saponins, terpenes, lipids, and carbohydrates.

Suitably the phenolics may be selected from simple phenolics, tannins, coumarins, flavonoids, chromones and xanthones, stilbenes, and lignans.

In certain preferred examples, the substance is at least one antisense oligonucleotide.

Suitably a plant growth regulator may be selected from auxins, cytokinins, ethylene, gibberellins, brassinosteroids, abscisic acid or other phytohormones. For example a growth regulator may be selected from 1-naphthalenacetic acid (NAA), 2,4-D, 3-indoleacetic acid (IAA), indolebutanoic acid (IBA), dicamba, picloram, gibberellic acid, 6-benzyl aminopurine (BAP), benzyl adenine (BA), 2-iP, kinetin, zeatin, dihydrozeatin, thidiazuron (TDZ), metatopolin, ethylene, florigen, abscisic acid (ABA), brassinosteroids (BR), jasmonic acid (JA), salicylic acid (SA), polyamines, strigolactones (SL) and nitric oxide (NO).

In certain examples, the plant growth regulator is gibberellic acid and/or DL-carnitine.

Suitably an epigenetic regulator may be selected from methyltransferase inhibitors, histone deacetylases and transferases, Cytosine Demethylation and DNA Glycosylases, Methylcytosine-Binding Proteins, Polycomb and Chromatin-Remodeling Proteins.

Suitably, an epigenetic regulator may be provided as a nucleic acid for expression in a plant.

Suitably epigenetic regulation may be provided by siRNA.

Suitably epigenetic regulation may be provided by a peptide.

Suitably an epigenetic regulator may be a small molecule epigenetic regulator.

Suitably an epigenetic regulator may be selected from 5-Azacytidine (5-aza) and 5-aza-2′-deoxycytidine (aza-dC), Trichostatin A, or sulfamethazine.

Suitably a peptide may be selected from an epigenetic regulator(s), plant defence peptide/protein(s), regulatory protein(s), for example regulatory proteins suitably to modulate plant developmental and physiological processes, transcription factor(s), flowering related protein(s) or the like.

Suitably a viral vector may be selected from RNA virus vector based on, for example Potato virus X (PVX), Tobacco rattle virus (TRV), Barley stripe mosaic virus (BSMV) and Cucumber mosaic virus (CMV) vectors, which are able to rapidly induce sequence-specific gene silencing through targeting the coding sequence or the promoter/regulatory sequences of a gene(s).

Suitably a volatile organic compound may be selected from small molecules with low boiling point and high vapour pressure, and may be an organic compound, suitably a synthetic organic compound selected from hydrocarbons, terpenes, alcohols, carboxylic acids and esters, ketones, or aromatics.

Suitably the VOC may be synthetically produced.

Suitably a VOC may be a plant VOC, a fungal VOC, a microbial VOC, combinations of plant VOCs, combinations of fungal VOCs or combinations of microbial VOCs, or combinations of at least two of a plant VOC, a fungal VOC and a microbial VOC.

Volatile organic compounds (VOCs) include numerous signaling molecules involved in plant-microbial interactions (Junker, R. R., and Tholl, D. (2013). Volatile organic compound mediated interactions at the plant-microbe interface. J. Chem. Ecol. 39, 810-825, Schulz-Bohm, K., Martin-Sánchez, L., and Garbeva, P. (2017). Microbial Volatiles: small molecules with an important role in intra- and inter-kingdom interactions. Front. Microbiol. 8:2484).

To date, a few thousand VOCs have been described in flowering plants (Knudsen, J. T., Eriksson, R., Gershenzon, J., and Ståhl, B. (2006). Diversity and distribution of floral scent. Bot. Rev. 72, 1-120) and microbes (Lemfack, M. C., Gohlke, B.-O., Toguem, S. M. T., Preissner, S., Piechulla, B., and Preissner, R. (2018). mVOC 2.0: a database of microbial volatiles. Nucleic Acids Res. 46, D1261-D1265). These VOCs predominantly include terpenoids, phenylpropanoids/benzenoids, fatty acids, and amino acid derivatives (Dudareva, N., Klempien, A., Muhlemann, J. K., and Kaplan, I. (2013). Biosynthesis, function and metabolic engineering of plant volatile organic compounds. New Phytol. 198, 16-32). Suitably the present invention may utilise such a plant VOC.

Suitably a plant VOC may be selected from β-caryophyllene, Ethylbenzene, D-Limonene, Cosmene, Cosmene (isomer), o-cymene, Methyl-heptenone, (z)-3-hexen-1-ol, Amyl ethyl carbinol, p-cymenene, Amyl vinyl carbinol, Furfurala-ionene, Dihydroedulan II, Dihydroedulan II, β-linalool, (R)-(+)-menthofuran, 5-methylfurfural, α-ionone, Hotrienol, trans-p-metha-2,8-dienol, Safranal, 3-furanmethanol, Tetramethyl-indane, Ethyl cyclopentenolone, p-menthen-1-ol, 4,7-dibenzofuran, Menthone, Camphor, 2-piperidin methenamine, 1-(1-butenyl)pyrrolidine, Methyl salicylate, trans-geraniol, Teresantalol, β-damascenone, 5-isoproprenyl-2-methylcyclopent-1-enecarboxaldehyde, Calamenene, Piperitenone, p-cymen-8-ol, Exo-2-hydroxy cineole, 3,6-dimethyl-phenyl-1,4-diol, Longipinene Isopiperitenone, Damascenone (isomer), Mint lactone, α,β-dihydro-β-ionone, Seudenone, Dihydroxy-durene, Cinerolon, Carvone, 1-acetoxy-p-menth-3-one, 2,6-diisopropyl naphthalene, (naphtalene derivative), Eugenol, 4-ethylphenol, Thymol, 2-acetyl-4-methylphenol, Carvacrol.

Suitably a fungal VOC may be selected from the Fusarium genus or Trichoderma. Saprophytic fungi, for example Cladosporium and Ampelomyces species (Kaddes A., Fauconnier M. L., Sassi K., Nasraoui B., Jijakli M. H. Endophytic fungal volatile compounds as solution for sustainable agriculture. Molecules. 2019; 24:1065, Morath S. U., Hung R., Bennett J. W. Fungal volatile organic compounds: a review with emphasis on their biotechnological potential. Fungal Biol. Rev. 2012; 26:73-83). Suitably a VOC may be selected from N-1-naphthylphthalamic acid (NPA). Suitably a VOC or multiple VOCs may be provided by said C. sphaerospermum selected from at least one of C. sphaerospermum Accession No. NRRL 67603, C. sphaerospermum Accession No. NRRL 8131, and C. sphaerospermum Accession No. NRRL 67749.

Suitably a VOC may be selected from γ-patchoulene, 3-methyl butanol, 1-octen 3-ol, 2-undecanone, 3-methylbutanoate, 2-methylbutan-1-ol, 4-methyl-2-heptanone, ethanethioic acid, 2-methyl propanal, ethenyl acetate, 3-methyl 2-pentanoene, methyl 2-methylbutanoate, methyl 3-methylbutanoate, 4-methyl 3-penten-2-one, 3-methyl 2-heptanone, myrcene, terpinene, methyl salicylate, 2-pentadecanone, 1H-pyrrole, ethyl butanoate, chlorobenzene, dimethylsulfone, 2-octanone, 5-dodecanone, 3-methyl-2-pentanone, geosmin, 1-pentanol, 2-methyl-1-propanol, dimethyl 2-octanol, disulfide, acetophenone, 2-isobutyl-3-methoxypyrazine, 2-heptanone, 5-methyl-3-heptanone, 2-methyl-2-butanol, 2-pentanol, 3-octanol, ethanol, anisole, 2-isopropyl-3-methoxypyrazine, hexanol, 2-methylfuran, 3-methyl-1-butanol, 2-pentanone, 3-octanone, 2-ethyl-1-hexanol, 1-butanol, isopropanol, 2-hexanone, 3-methylfuran, 3-methyl-2-butanol, 2-pentylfuran, 1-octen-3-ol, 2-ethylfuran, 2-butanone, isopropyl, 3-hexanone, acetate, isobutyrate, 2-methylisoborneol, isovaleraldehyde, a-terpineol, 2-nonanone, ethylfuran, 2r,3r-butanediol, 2-methyl-1-butanol, citric acid, 1-octanol, a Nod factor, a flavonoid, a strigalactone, or any combination or derivative thereof.

Suitably a VOC(s) can be injected into a gas flow for incorporation into a micro- and/or nanobubble or to provide the VOC(s) in combination with or in solution with a micro- and/or nanobubble.

Suitably a compound capable of inducing a change in the phenotype, chemistry, or physiology of a plant may enter into the micro- and/or nanobubble as the liquid solution containing the compound(s) is recirculated through a nanobubble generator.

Suitably a compound capable of inducing a change in the phenotype, chemistry, or physiology of a plant may bind to the surface of a micro- and/or nanobubble.

In methods of the invention which include a plant pre-treatment step, a plant root can be prepared to allow greater uptake of the gas or gases in the micro and/or nanobubble.

Suitably a root portion can be cleaned to allow uptake.

Suitably a root portion may be pre-oxygenated before the mixture of micro- and/or nanobubbles with one or more compound discussed herein, for example, a nucleic acid, a plant epigenetic regulator or VOC, is applied.

Suitably a pre-treatment step can comprise incubating rooted shoots in a nanobubble water (or other suitable liquid medium) formed using a gas for example comprising or consisting of air, oxygen, carbon dioxide, or another suitable gas or combinations thereof, suitably oxygen nanobubble water/liquid medium.

Suitably, pre-treatment may be provided for at least one, at least two days, at least one week, at least one month, or several months prior to treatment with the mixture comprising a compound(s).

As will be appreciated, pre-treatment may be suitably applied in view of the size, health, growth stage or other condition of the root zone. It is considered a suitable pre-treatment step may lead to improved uptake of a compound or compounds.

Suitably the methods of the invention can be undertaken in real time, to allow uptake of one or more compounds discussed herein, for example a nucleic acid, a plant epigenetic regulator or VOC, to be provided at any time in the life of the plant.

Suitably, the combination of oxygen nanobubble water/liquid medium and a compound or compounds to alter gene expression can be done at any time in the plant's life cycle to effect changes in real time.

Suitably the uptake of the compound may also be monitored in real time to allow control of delivery of the micro and/or nanobubble and compound mixture.

Suitably the micro and/or nanobubble and compound mixture may be provided to the plant via a standard dripper to the root of the plant, for example delivery of the micro and/or nanobubble and compound mixture by standard watering or irrigation systems.

Suitably delivery may be to soil, aquaponics systems, standard plant growing media, coco coir, coir, coco peat, standard plant tissue growing substrates or media, or other non-soil substrates.

Suitably the plant epigenetic regulator to provide covalent modifications of DNA and/or histones, affecting transcriptional activity of chromatin without changing DNA sequence may induce DNA methylation, RNA methylation, histone methylation or histone acetylation. For example siRNA can induce DNA methylation. The epigenetic regulator can induce transient changes which could last a short time (hours, days, or weeks), or could last the lifetime of the plant.

Suitably a plant epigenetic regulator may induce DNA methylation, RNA methylation, histone methylation or histone acetylation in one or more flowering loci.

Suitably the epigenetic regulator may be selected from a volatile organic compound (VOCs), siRNA, other RNAs, antisense oligonucleotides, plant growth regulators, peptides, RNAi vectors, expression vectors and/or viral vectors.

The mixture of nano- or microbubbles and compounds can be used along with transgenes, gene editing vectors, RNAi vectors, expression vectors or viral vectors to enhance uptake into recalcitrant plant cells via the roots or other germline tissues.

Suitably viral vectors may comprise nucleic acids for gene silencing or to enhance gene expression, for example transient gene expression via exogenous nucleic acids, for example exogenous genes which may be expressed to provide a product of interest.

Suitably the mixture may further comprise one or more further constituents such as surfactants, chelating agents, emulsifiers, antioxidants, reductants, pH and osmotic buffers.

Suitably, the compound may be a plant epigenetic regulator that induces DNA methylation, RNA methylation, histone methylation or histone acetylation to provide a heritable change.

Suitably a plant epigenetic regulator may induce DNA methylation, RNA methylation, histone methylation or histone acetylation in one or more flowering loci.

Suitably the epigenetic regulator may be selected from a volatile organic compound(s) (VOC(s)), siRNA, other RNAs, antisense oligonucleotides, plant growth regulators, peptide, RNAi vectors, expression vectors and/or viral vectors.

Suitably the microbubbles and/or nanobubbles may be generated using one or a mixture of gases. For example the gas may be selected from the group comprising or consisting of air, oxygen, carbon dioxide, nitrogen, hydrogen, ethylene, ethylene oxide and combinations thereof.

Suitably the microbubbles or nanobubbles may be generated in the presence of oxygen to provide an oxygen-enriched liquid, which may then be applied to plant roots.

Suitably the microbubbles or nanobubbles may be provided by any method as known in the art including swirl-type liquid flow, venturi, high-pressure dissolution, ejector, mixed vapour direct contact condensation, electrical field and supersonic vibration.

For example, spinning a liquid around a motor, raising the flow rate of a liquid by pump pressure; providing air or another gas or gasses to the liquid; and stirring the liquid to provide bubbles and then disrupting the bubbles to form microbubbles, or nanobubbles may be used.

Alternatively, air or other gas or gasses via a jetting nozzle may be provided to a liquid such that bubbles jetted from the jetting nozzle are torn into super-micro bubbles by the force of jet flow of the liquid jetting nozzle.

Further alternatively, bubbles may be generated by stirring, and then passing the generated bubbles through the eyes of a mesh membrane to form nanobubbles.

Yet further alternatively, a compressor for delivering gas under pressure into liquid and a bubble generation medium may be provided, wherein the bubble generation medium consists of a high-density compound which is an electrically conductive substance. By jetting liquid in a direction substantially perpendicular to the direction in which the bubble generation medium discharges, nanobubbles may be generated as described in EP 2 460 582.

Suitably combinations of these methods or other methods known in the art may be utilised.

Suitably a nanobubble refers to a bubble that has a diameter of less than one micron. A microbubble, which is larger than a nanobubble, is a bubble that has a diameter greater than 1 micrometre in diameter.

Suitably at least 50% of the nanobubbles generated have a diameter of less than 300 nm, suitably 80 nm or less, optionally 20 nm or less.

Suitably a nanobubble may have a mean diameter less than 500 nm or less than 200 nm, or ranging from about 20 nm to about 500 nm (e.g., from about 75 nm to about 200 nm).

Suitably a microbubble or nanobubble mixture may be provided, for example a micro- or nanobubble with a bubble diameter of 200 nm-10 μm.

Most conventionally-formed bubbles in a liquid easily float to the water surface, burst and the gas contained in the bubble merges with the atmosphere above the liquid. In contrast, nanobubbles may be only slightly affected by buoyancy and exist as they are in the liquid for a longer period of time. Suitably a nanobubble as used in the present invention may have a lifetime of at least one hour, at least 2 hours, at least 3 hours, at least 5 hours, at least 1 day, at least 1 week, for at least one month or for at least three months under ambient pressure and temperature. Suitably a nanobubble may have high gas solubility into the liquid due to its high internal pressure.

Suitably the nanobubbles may be positively or negatively-charged nanobubbles. For example the nanobubbles may have a zeta potential of 10 mV to 200 mV, or −10 mV to −200 mV. Suitably the nanobubbles may have a zeta potential of 5 mV to 150 mV, or −5 mV to −150 mV. Suitably stability of the nanobubbles may be provided due to negatively charged surfaces of the nanobubble. Suitably pH may be used to generate charged micro-nanobubbles. Suitably electrical fields may be used to provide and/or change the zeta potential of micro- and/or nanobubbles

Suitably a concentration of nanobubbles in a liquid carrier may be at least 10E+05 bubbles per ml, for example as determined using a Zetasizer (Zetasizer Ultra) or other suitable apparatus.

Suitably, the plant application medium is provided to a plant for an application period of at least 1 hour, at least 4 hours, at least 12 hours, at least 24 hours, at least 48 hours, at least 7 days, at least 10 days, at least 14 days, at least 20 days, or over the lifetime of the plant, optionally over the cultivation duration of the plant.

Suitably, the plant application medium is provided to a plant for at least 1 hour each day, at least 4 hours each day, at least 12 hours each day, or continuously each day over the application period.

Suitably, the plant application medium is provided to a plant at less than 1 hour post-germination, at 1, 2, 3, 4, 5, 6, 7, 8, 9, 10, 11, 12, 13, 14, 15, 16, 17, 18, 19, 20, 21, 22, 23, or 24 hours post-germination, or at 1, 2, 3, 4, 5, 6, 7, 8, 9, 10, 15, 20, 25, or 30 days post-germination, or at 1, 2, 3, 4, 5, 6, 7, 8, 9, 10, 11, or 12 months post-germination, or at 1, 2, 3, 4, 5, 6, 7, 8, 9, 10, or more years post-germination. Germination is considered to occur with the emergence of the root and cotyledonary leaves.

Suitably as discussed herein, a plant may be considered leaf plants, fruit plants, grains and algae, or mosses.

Suitably a plant may be a seed or another plant part, such as a leaf or leaf section, a piece of stem, pollen, anther, embryo, or any other stem cells of the plant from which new plants can be grown.

Suitably a plant tissue (explant) may be incubated on solid media containing nanobubbles and compounds to enhance uptake of transformation vectors, etc. into recalcitrant plant species.

Suitably, the plants used may be selected from the group comprising higher or vascular plants adapted to synthesise metabolites in a large quantity. Suitably a plant may include Cannabis, hemp, maize/corn, soy, rice, wheat, potato, sugarcane, arbuscular mycorrhiza fungi, tomato, lettuce, microgreens, cabbage, barley, tobacco, pepper, sorghum, cotton, sugar beets, or any other legumes, fruits, nuts, vegetables, pulses, flowers, or other commercial crop not inconsistent with the objectives of this disclosure.

Suitably, a plant may be selected from, without limitation, energy crop plants, plants that are used in agriculture for production of food, fruit, wine, biofuels, fibre, oil, animal feed, plants used in the horticulture, floriculture, landscaping and ornamental industries, and plants used in industrial settings.

Suitably a plant may comprise gymnosperms (non-flowering) or angiosperms (flowering). If an angiosperm, the plant can be a monocotyledon or dicotyledon. Non-limiting examples of plants that could be used include desert plants, desert perennials, legumes, such as Medicago sativa, (alfalfa), Lotus japonicas and other species of Lotus, Melilotus alba (sweet clover), Pisum sativum (pea) and other species of Pisum, Vigna unguiculata (cowpea), Mimosa pudica, Lupinus succulentus (lupine), Macroptilium atropurpureum (siratro), Medicago truncatula, Onobrychis, Vigna, and Trifolium repens (white clover), corn (maize), pepper, tomato, Cucumis (cucumber, muskmelon, etc.), watermelon, Fragaria, other berries, Cucurbita (squash, pumpkin, etc.) lettuces, Daucus (carrots), Brassica, Sinapis, Raphanus, rhubarb, sorghum, miscanthus, sugarcane, poplar, spruce, pine, Triticum (wheat), Secale (rye), Oryza (rice), Glycine (soy), cotton, barley, tobacco, potato, bamboo, rape, sugar beet, sunflower, peach (Prunus spp.) willow, guayule, eucalyptus, Amorphophallus spp., Amorphophallus konjac, giant reed (Arundo donax), reed canarygrass (Phalaris arundinacea), Miscanthus giganteus, Miscanthus sp., sericea lespedeza (Lespedeza cuneata), millet, ryegrass (Lolium multiflorum, Lolium sp.), Phleum pratense, Kochia (Kochia scoparia), forage soybeans, Cannabis, hemp, kenaf, Paspalum notatum (bahiagrass), bermuda grass, Pangola-grass, fescue (Festuca sp.), Dactylis sp., Brachypodium distachyon, smooth bromegrass, orchard grass, Kentucky bluegrass, turf grass, Rosa, Vitis, Juglans, Trigonella, Citrus, Linum, Geranium, Manihot, Arabidopsis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Browaalia, Phaseolus, Avena, Hordeum, and Allium.

Suitably the phenotype, chemistry, or physiology of a plant is altered to enhance the production of a plant-based pharmaceutical and/or industrial product, medicinal and non-medicinal health-related or recreational products, neutraceutical or other functional food product, cosmetical compound, additive, bioceutical, or agricultural product provided by the plant or components used in these fields.

Suitably, engineering the expression of a secretory pathway or parts thereof in plants enables the production of molecules for example biologics that would otherwise accumulate at low levels or in an improperly processed form.

Suitably an enhanced product may be, but is not limited to, a phytohormone, a flavonoid, in particular chalcones, flavones, flavonols, flavandiols, anthocyanins, and proanthocyanidins, condensed tannins or aurones.

Suitably an enhanced product may be a sugar substitute, for example steviol glycosides. For example the enhanced product may comprise or consist of Stevioside, Rebaudioside A, Rebaudioside C, Dulcoside A, Rebaudioside B, Rebaudioside D and/or Rebaudioside E.

Suitably an enhanced product may be a plant-derived pharmaceutical, for example a cardiotonic Acetyldigoxin, Adoniside, Convallatoxin, Deslanoside, Digitalin, Digitoxin, Digoxin, Etoposide, Gitalin, Lanatosides A, B, C, Ouabain.

Suitably, the enhanced product may be an anti-inflammatory, for example Aescin.

Suitably, the product may be an anticholinergic—Anisodamine, Anisodine, Atropine, Hyoscyamine. Suitably, the product may be an anti-cancer—Betulinic acid, Camptothecin, Colchicine amide, Colchicine, Demecolcine, Irinotecan, Lapachol, Monocrotaline, or Taxol.

Suitably, the plant product may be selected from Aesculetin, Agrimophol, Ajmalicine, Allantoin, Allyl isothiocyanate, Anabesine, Andrographolide, Arecoline, Asiaticoside, Benzyl benzoate, Berberine, Bergenin, Borneol, Bromelain, caffeine, Camphor, (+)-Catechin, Chymopapain, Cissampeline, Cocaine, Codeine, Curcumin, Cynarin, Danthron, Deserpidine, L-Dopa, Emetine, Ephedrine, Galanthamine, Glaucarubin, Glaucine, Glasiovine, Glycyrrhizin, Gossypol, Hemsleyadin, Hesperidin, Hydrastine, Kaibic acid, Kawain, Kheltin, Morphine, Papavarine, Pilocarpine, Sanguinarine, Scopolamine, Silymarin.

Suitably the product may be a plant-derived cancer drug, for example vinca alkaloids (vinblastine, vincristine and vindesine), epipodophyllotoxins (etoposide and teniposide), taxanes (paclitaxel and docetaxel) or camptothecin derivatives (camptotecin and irinotecan).

Suitably an enhanced product may be a compound naturally formed by a plant, such as cannabis. In particular, one or more epigenetic regulators may be supplied to the plant as described herein to modulate a single or groups of metabolic pathways. This can modify the profile of compounds normally expressed to create a platform for cannabis that provides a route to commercial-scale quantities of the common component cannabidiol and to lesser compounds such as methyl-(C₁), butyl-(C₄) and other Cₙ alkyl cannabinoids.

Suitably modulation of latent biosynthetic pathways in the plant can be utilised to create new pharma based on the aforementioned cannabis molecules but with chemistries altered through glycosylation, o-alkylation, esterification, acetylation, terpene addition and ionisation through addition of inorganic moieties (phosphate, sulphate, nitrate and ammonium).

Suitably an enhanced product may be a colourant. For example, many plants, e.g. Empetrum nigrum, and Isatis tinctoria and Crocus sativus, produce colours used in food, textile, hair dyes etc. Using the processes as described herein, the diversity and proportion of compounds provided by a plant can be modulated to create sustainable colourant feedstocks (crops) with specific (visible spectrum) and reproducible colours. Furthermore, triggering of latent pathways may be utilised to alter the chemistries of the colourants thereby expanding their utility through alkylation, specific oxidation/reduction, glycosylation to provide functional differences such pH stability, photodegradation, water/oil solubility etc.

Suitably an enhanced product may be a functional molecule, for example, a surfactant. Surfactants are organic compounds used to mix two immiscible substances, such as oil and water. They are used in many industries worldwide, most notably those of cosmetic, healthcare, and food and drink. A significant fraction of the market demand for surfactants is met by organo-chemical synthesis using petrochemicals as precursors. The methods disclosed herein may be utilised with seed crops (such as oilseed rape (or other Brassicaceae)), to enhance yield of galactolipids, known as sustainable emulsifiers. Epigenetic regulator application enables new emulsifier/surfactant chemical variants (oligogalactolipids) and an increase in yield, particularly of lesser known/modified galactolipids. Alternatively, emulsifier specific activity, stability/durability, functional pH range etc. may be altered by altering the pathways within the plant utilising the methods disclosed herein.

Suitably an enhanced product may be a functional food molecule, for example egg replacer, such as egg albumin replacer. Such a functional food molecule may act as an emulsifier, clarifier, textural modification, binder, nutritional component, stabiliser, glazing agent etc. Egg albumin is a member of the serpin super family of protease inhibitors. Serpins existing in many plants such as barley where Protein Z is abundant in the grain and the methods of the present invention can be utilised to increase the yield of such serpins.

Suitably the phenotype or physiology of a plant may be altered to enhance a structural growth characteristic of the plant. For example a structural growth characteristic may be selected from growth rate, biomass weight (whole plant, aerial portion of plant, root, tuber), plant height, number of branches, branch thickness, branch length, branch weight, number of leaves, leaf size, leaf weight, leaf thickness, leaf expansion rate, petiole size, petiole diameter, petiole thickness, stem thickness, trunk thickness (caliper), stem length, trunk length, stem weight, trunk weight, canopy/branching architecture, root biomass, root extension, root depth, root weight, root diameter, root robustness, root anchorage, or root architecture.

Suitably the phenotype or physiology of a plant may be altered to enhance a growth characteristic of the plant in response to environmental conditions for example selected from abiotic stress tolerance such as cold, heat, salinity and/or drought), or in response to biotic stress from for example microbial or fungal attack or infestation or predation.

Suitably the phenotype or physiology of a plant may be altered to enhance a growth characteristic of the plant selected from: anthocyanin pigment production, anthocyanin pigment accumulation, plant oil quality and quantity, secondary metabolite accumulation, sensory and flavour compound production, content of phytopharmaceutical or phytochemical compounds, protein content, fibre hypertrophy and quality, quantity of chlorophyll, photosynthesis rate, photosynthesis efficiency, leaf senescence retardation rate, early and efficient fruit set, early fruit maturation, fruit yield, yield of vegetative parts, root and tubers, fruit/grain and/or seeds, size of fruit, grain and/or seeds, firmness of fruit, grain and/or seeds, weight of fruit, grain and/or seeds, starch content of vegetative parts, root and tuber, fruit, grain, and/or seeds, sugar content of fruit, grain and/or seeds, content of organic acids in fruit and seeds, early flowering (flowering precocity), or harvest duration.

Suitably the phenotype or physiology of a plant is altered to enhance a combination of growth characteristics of the plant.

Suitably the plant may be grown on or in soil-less culture on a porous support, by continuous soaking in the nutrient solution, by temporary immersion in said nutrient solution (sub-irrigation, hydroponics, nutrient film, etc.), by use of a standard dripper, or by contacting with a nutrient solution in the form of a mist or fog (aeroponics).

Suitably, in the processes of the invention, the mixture is applied to an organ of the plant.

Optionally, the organ is the plant root system. Suitably, the mixture is applied to the plant root system via a liquid plant growth medium.

Nanobubbles as discussed herein are considered to provide several unique physical and mechanical characteristics. For example they provide longevity, virtual disappearance of buoyancy, high internal pressure, extremely large surface/volume ratio, high oxygen dissolution rate.

Suitably VOCs may be injected into the air/gas channel going into the nanobubble generator to provide these in combination with the nanobubble. Suitably, other compounds may be introduced into the nanobubble water/liquid carrier which is recirculated through the nanobubble generator to provide such compounds in the methods of the invention.

Suitably a nanobubble generator may comprise a compressor for delivering gas under pressure, a bubble generation medium for discharging the gas, which has been delivered under pressure, as super-micro bubbles into liquid, wherein the bubble generation medium consists of a high-density compound which is electrically conductive.

The nanobubble generator may also be provided with a liquid jetting device for jetting liquid in the direction substantially perpendicular to the direction in which the bubble generation medium discharges the bubbles. The jetting liquid may be the same kind of liquid as the liquid into which the super-micro bubbles are discharged. EP 2460582 and U.S. Pat. No. 8,919,747 describe nanobubble generators which are suitable for use in the processes and systems of the present invention.

Suitably a compound may be delivered into liquid. This liquid may be constantly recirculated through the nanobubble generator. As the liquid pushes the nanobubbles out through a surface there can be some coalescence as bubbles reform before they leave the surface. At that stage the compounds may be taken up inside the nanobubble or attached to its surface.

Suitably nanobubbles may be provided under sterile conditions. The apparatus (gas supply, recirculating liquid culture media, water, sugar solution or other liquid medium and nanobubble generator) may be housed in, and the processes carried out in, a laminar flow cabinet. Suitably liquid or solid media containing nanobubbles are produced for tissue culture.

Suitably there is provided a system wherein the system is sterile and/or automated.

Suitably a system as described herein, wherein the step of applying the mixture to a plant comprises applying the mixture to a plant in a hydroponic plant cultivation system.

### DETAILED DESCRIPTION

The above and other aspects of the present invention will now be described in further detail, by way of example only, with reference to following examples and the accompanying figures, in which:

FIG. 1 illustrates uptake of CY3-labelled DNA oligo in various plant tissues with or without oxygen nanobubbles after 24 or 30 hr incubation of roots in either oxygenated (0) water, water or oxygen nanobubble (ONB) water. CY3 was visualised using a LSM 710 upright confocal microscope. a) Cannabis sativa (Cs) root after 30 hr; b) Cs leaf after 30 hr, 10× magnification; c) Nicotiana benthamiana (Nb) leaf after 24 hr, 10× magnification; d) Hordeum vulgare (Hv) leaf after 24 hr, 10× magnification; e) Ocimum basilicum (Ob) leaf after 24 hr, 10× magnification.

FIG. 2 illustrates uptake of CY3 labelled phytoene desaturase (PDS) oligo in Cannabis sativa (Cs) plant tissues after 3 or 30 hr incubation of roots in tap water or with oxygen nanobubbles (ONB) using a LSM 710 upright confocal microscope. a) Cs root after 30 hr; b) Cs leaf after 3 hr, 10× magnification; c) Cs leaf after 30 hr, 20× magnification; with ONB treatment compared to water without ONB.

FIG. 3 illustrates the range of plant material used for DNA oligo treatment. a) Cannabis sativa (Cs) rooted plants in 50 ml falcon tubes; b) Cs rooted cutting in Eppendorf; c) Cs rooted cuttings in coco coir; d) Nicotiana benthamiana (Nb) seedlings in eppendorfs. e) Hordeum vulgare (Hv) seedling in universal tube. A range of ages from 3-6 weeks old were used.

FIG. 4 illustrates phenotype in a) Cannabis sativa (Cs) leaf 5 days after incubation with PDS antisense oligos; b) Hordeum vulgare (Hv) leaves 20 days after incubation with PDS antisense oligos (right). Control plants without ONB (left); c) Nicotiana benthamiana (Nb) leaves 20-37 days after incubation with PDS antisense oligos

FIG. 5 illustrates PDS mRNA levels in a) Cannabis sativa (Cs) leaf 5 days after incubation with PDS antisense oligos; b) Nicotiana benthamiana (Nb) leaf 37 days after incubation with PDS antisense oligos in water or ONB water. mRNA levels quantified by qPCR relative to eF1a control.

FIG. 6 illustrates the size distribution of nanobubbles measured in ONB water prepared for antisense oligo treatments. Size distribution was measured using a Zetasizer Nano ZS. Size of nanobubbles were stable for over 12 days.

FIG. 7 illustrates the effect of oxygen nanobubbles (ONBs) on Agrobacterium strain AGL1 uptake by Nicotiana benthamiana (Nb) seedlings. a) Nb seedling roots were incubated in MS30 liquid medium containing Agrobacterium expressing GUS under a constitutive promoter with ONB (left) or without (right) and a control without Agrobacterium (middle) for two days prior to staining for GUS activity. b) Nb seedlings immersed in ¼ MS10 medium containing Agrobacterium expressing a GUS version containing an intron with ONB (left) or without (right) for four days prior to staining for GUS activity.

FIG. 8 illustrates the effect of oxygen nanobubbles (ONBs) on CRISPR/Cas9 based gene editing efficiency. Agrobacterium containing CRISPR/Cas9 construct was introduced into seedlings of a Nicotiana tabacum (Nt) transgenic line containing a non-functional 13-Glucosidase (GUS) repeat as a CRISPR target gene. Upon DNA break induction by Cas9/gRNA, homologous recombination (HR) between the two fragments restores the functional GUS gene. Strong HR was detected as blue staining in the presence of both ONBs and Agrobacterium containing CRISPR construct targeting GUS gene. Tap_C=tap water and Agrobacterium containing CRISPR construct targeting a potato gene (control); Tap_CRISPR=tap water and Agrobacterium containing CRISPR construct targeting GUS gene; ONBs_C=ONBs and Agrobacterium containing CRISPR construct targeting a potato gene (control); ONBs_CRISPR=ONBs and Agrobacterium containing CRISPR construct targeting GUS gene. * ONBs_CRISPR seedlings contained additional patches of GUS that were not quantifiable as single spots so not included in the graph.

FIG. 9 illustrates an example of a plant cultivation system as discussed herein.

FIG. 10 illustrates the use of nanobubbles as a delivery system for volatile compounds to improve growth in Ocimum basilicum (Ob) seedlings. a) Ob seedlings after 21 days growing in nanobubble hydroponic system with and without a volatile compound; b) Effect of volatile introduced with nanobubbles on a number of growth parameters in Ob seedlings.

FIG. 11 illustrates the optimisation of delivery methods for introducing nanobubbles and volatile compounds to improve growth in Ocimum basilicum.

FIG. 12 illustrates the use of nanobubbles as a delivery system for liquid feed (Canna Coco A+B) to improve growth in Cannabis sativa. Plant biomass was significantly greater when liquid feed was introduced with oxygen nanobubbles (ONB) compared to control water with no nanobubbles.

FIG. 13 illustrates the use of nanobubbles as a delivery system for Plant Growth Regulators to improve plant growth in Cannabis sativa. Increased plant height and biomass when using ONB compared to tap water as a delivery system for a) gibberellic acid; and b) DL-carnitine.

FIG. 14 illustrates the effect of ultrasonic fogging of leaves with air nanobubbles containing volatile compound on growth of Lactuca sativa varieties.

### EXAMPLES

The following examples use a AZ-FB-20ASW nanobubble generator obtainable from Anzaikantetsu Co—http://anzaimcs.com/en/main/examplenanobubble.html. FIG. 9 illustrates an example of a system comprising immersion of roots in a nanobubble medium, which is in fluid communication with a nanobubble generator. It will be appreciated that alternative or additional systems could be arranged to apply the medium to the plant.

All materials were obtained from commercial suppliers.

### Example 1

In an initial experiment three different water treatments were set up to compare efficiency of antisense oligo transfer to the plant cells via the roots.
1. Oxygenated tap water (O water) was prepared by bubbling oxygen through an air curtain into water at very low pressure. The dissolved oxygen in this water averages 300% air saturation.
2. Standard tap water was used as a control. The dissolved oxygen in tap water averages 100% air saturation.
3. Oxygen nanobubble tap water (ONB) was prepared by continuous feed from an oxygen cylinder into a nanobubble machine at 2 bar pressure with standard tap water being fed through the nanobubble machine to collect oxygen nanobubbles. The dissolved oxygen in nanobubble water averages 400% air saturation.

All water treatments were circulating independently in troughs.

The roots of Cannabis sativa plants were pre-treated in each of the water treatments for 30-120 mins prior to transfer into 50 ml falcon tubes along with 5 ml samples from their respective troughs as shown in FIG. 3a. CY3 labelled DNA antisense oligo was added to each water treatment to give a final concentration of 1 uM. to each plant from each water treatment. All samples were stored at room temperature in the dark before removal of root and leaf sections at 3 hrs and 30 hrs for visualization under a LSM 710 upright confocal microscope. FIGS. 1a, 1b show results after 30 hrs incubation. Uptake and transport of antisense oligos incubated in ONB water was significantly higher than with other water treatments. Treatment with oxygenated water shows that increased uptake of antisense oligos was due to the presence of nanobubbles rather than oxygen in the water. Substituting tap water with ultrapure water in all treatments gave similar results.

### Example 2

A series of experiments were performed to demonstrate the uptake of antisense oligos in plants, rooted cuttings or seedlings (FIG. 3 a-e) from a number of plant species through introduction at the roots with and without oxygen nanobubbles (ONB).

Fluorescence was measured in leaves 24 hrs hrs after CY3 labelled antisense oligos were introduced indicating efficient transport of antisense oligos from roots to leaves (FIG. 1 c-e). Significantly higher fluorescence signal was visible in all leaves sampled from ONB treatments compared to control treatments.

In further experiments antisense oligos were introduced to silence the phytoene desaturase (PDS) gene with or without CY3 labelling in Cannabis sativa (Cs). Fluorescence was visualised 3 hrs or 30 hrs after introduction of the antisense oligos under confocal (FIG. 2a-c). Silencing of the PDS gene leads to albino phenotype in leaves due to impairment of carotenoid and chlorophyll biosynthesis. Albino leaf phenotype was visible in a number of plant species (FIG. 4a-c) and further quantified by real-time quantitative PCR (qPCR) to determine the reduction of mRNA levels in leaves (FIG. 5a-b). In a series of experiments levels of PDS mRNA were reduced by up to 80% using antisense oligos combined with ONB.

It was considered the optimal size range for oxygen nanobubbles used to transport compounds through plant roots was 10 nm-150 nm. The nanobubble water generated was found to be stable for days, possibly weeks (FIG. 6).

The combination of nanobubbles and compounds introduced to the plant in combination have proven to be a fast, effective way to induce changes in gene expression. In contrast to oxygenated water where the fluorescence signal is low and the tap water where the fluorescence signal is mainly in the trichomes, with ONB the signal is present in the majority of leaf cells. This provides a highly efficient system to effect change(s) in real time such as inducing flowering which has to be done in a fully grown plant.

### Example 3

A series of experiments were done to introduce Agrobacterium tumefaciens strain AGL1 cells containing vectors expressing the β-Glucosidase (GUS) gene in Nicotiana benthamiana (Nb) seedlings to compare uptake with and without oxygen nanobubbles (ONBs). First, Nb seedlings at the 2-leaf stage were incubated with Agrobacterium containing a construct with GUS under control of a constitutive promoter (FIG. 7a) in MS30 liquid made with or without ONB water and with or without Agrobacterium and incubated for 2 days prior to staining for GUS activity. Higher expression of GUS was detected in seedlings incubated with MS30 made with ONB water compared to treatments without ON B water.

In a further experiment four-week-old Nb seedlings were transformed with AGL1 containing a transgene construct with β-Glucosidase (GUSPIus) gene with an intron (black line) under the transcriptional control of Arabidopsis ubiquitin 10 promoter (AtUB110p) and the terminator of tobacco extensin (NtExtT) (FIG. 7b). The seedlings were immersed in ¼ MS10 medium containing Agrobacterium with or without ONBs and incubated for four days prior to staining for GUS activity. Higher expression of GUS was detected in seedlings incubated with ONB water compared to treatment without ONB water.

A further experiment was done to determine the effect of oxygen nanobubbles (ONBs) on CRISPR/Cas9 based gene editing efficiency. A CRISPR/cas9 construct was made to express tomato-codon-optimised Cas9 (LeCas9) and a guide RNA (gRNA) to target β-Glucosidase (GUS) gene (FIG. 8a). The target GUS gene is made of two defective partial GUS fragments missing the 5′ or 3′ end and separated by the selectable marker hygromycin (hpt) (FIG. 8b). This transgene was introduced into Nicotiana tabacum (Nt) SR1 to generate transgenic lines (GUS^{DR}) where rare spontaneous homologous recombination (HR) events can be detected as blue spots (FIG. 8c, white arrow). The transformation of GUS^{DR} seedlings with Agrobacterium strain AGL1 containing CRISPR/Cas9 construct will result in DNA break at one or both sites (red triangle) in the overlapping GUS region. The double-strand break repair by HR results in the restoration of a functional GUS. Such recombination events can be detected by histochemical staining for GUS activity showing blue spots on seedlings (FIG. 8d, e, white arrows and enlarged areas (white boxes)). The number of blue spots per seedling was scored for each treatment (FIG. 8f). Strong HR was detected in the presence of both ONB and Agrobacterium containing CRISPR_GUS construct targeting GUS gene. The samples are Tap_C, tap water and Agrobacterium containing CRISPR construct targeting a potato gene (control); Tap_CRISPR, tap water and Agrobacterium containing CRISPR construct targeting GUS gene; ONBs_C, oxygen nanobubbles and Agrobacterium containing CRISPR construct targeting a potato gene (control); ONBs_CRISPR, ONBs and Agrobacterium containing CRISPR_GUS construct targeting GUS gene. This HR assay probably underestimates the efficiency of gene editing since other expected insertion/deletion (Indel) events are not detectable by GUS staining. This experiment shows higher efficiency of CRISPR/Cas9 based gene editing with ONB water.

### Example 4

It is understood that bacteria and fungi can promote plant growth through mutualistic interactions involving volatile organic compounds. Cladosporium sphaerospermum strain TC09 has been shown to enhance plant growth through the release of VOCs taken up by the plant tissues in vitro.

It is considered a nanobubble generator can be fluidly connected to a hydroponic system to feed nanobubbles containing VOCs into the hydroponic system (plant growing system). As an example, VOCs from TC09 (for example from C. sphaerospermum, in particular wherein said C. sphaerospermum is at least one of C. sphaerospermum Accession No. NRRL 67603, C. sphaerospermum Accession No. NRRL 8131, and C. sphaerospermum Accession No. NRRL 67749) growing in a container can be provided along with oxygen (or other gas from carbon dioxide, nitrogen, air) into a gas inlet of a nanobubble generator. Water can be pumped through the nanobubble generator to produce nanobubble water containing oxygen and VOCs. The produced nanobubble water containing oxygen and VOCs can then be circulated/re-circulated around plant roots, for example using any appropriate plant growing system.

Suitably nanobubble water with at least one compound that induces a change in the phenotype, chemistry or physiology of a plant can be re-circulated in a hydroponic system with the plants. As will be appreciated, nanobubble water may comprise other nutrients or the like to provide a liquid medium that may be provided to a plant. Several potential set ups can be utilised to provide nanobubble water to a plant for example plants can be provided in troughs, wherein the troughs are part of a recirculating system with water constantly moving over plant roots. Alternatively, the plants can be provided in a set up wherein the nanobubble water is provided as part of an ebb and flow system where pots are filled and emptied intermittently as nanobubble water is pumped through the system.

A series of experiments using volatiles were conducted to determine the efficiency of using nanobubbles as a delivery system.

A volatile compound was introduced via evaporation into a gas line (FIG. 9) feeding into a nanobubble generator in a recirculating water system for 21 days. Ocimum basilicum (Ob) seedlings growing in this volatile nanobubble water had greater plant heights, shoot wet biomass, number of stems, number of leaves and weight of leaves compared to basil seedlings growing in nanobubble water without volatiles (FIG. 10). This experiment shows nanobubbles are efficiently transporting the volatiles to the plants through the roots.

Further experiments were conducted to optimise the delivery method of volatiles with nanobubbles to plants through the roots in hydroponics with recirculating water. Different concentrations of volatiles and two methods of preparation of volatiles with plant feed and ONB were tested. In the first method, ONB water was prepared; then plant liquid feed (in concentration that was optimal for plant growth in tap water) and different concentration of volatiles were added to the ONB. In the second method, the volatiles and liquid feed mixtures were prepared and then were run through a nanobubble generator. The second method of nanobubble preparation proved to deliver the liquid feed and the volatiles more efficiently. The control plant growth from the second set up was inhibited by the concentration of the nutrients (FIG. 11), indicating that it is possible to reduce concentration of the fertiliser to be used in the hydroponics trials when delivered with nanobubbles. Additionally, the second NB preparation method showed a dose response to volatiles, proving the volatiles were transported through roots to the plants in the presence/inside the nanobubbles generated. The liquid feed concentration was better tolerated by plants when even small amounts of volatiles were added to the water solutions before the nanobubbles were generated. This indicates those volatiles improved plant growth under salt stress conditions.

### Example 5

A series of experiments were performed to demonstrate the efficient uptake of liquid nutrients with nanobubbles in Cannabis sativa (Cs). Two water treatments were set up to compare transfer of the liquid feed to the plant roots: 1) Standard tap water was used as a control and 2) Oxygen nanobubble tap water (ONB). The hydroponic experiments were set up in glasshouse conditions: day temp. 25° C., night temp. 18° C., 16/8 h day/night and 150 μmol m⁻² s⁻¹ light intensity. Cs apical cuttings were used. Oxygen nanobubble tap water (ONB) was generated using a fine bubble generator (Anzaikantetsu Co, AZ-FB-20ASVV) with a 0.75 standard litres per minute (SLPM) O₂ flow and 800 L/H water flow. 120 L of tap water was run for 3 hrs through the nozzle. Next, different treatments were prepared in 25 L buckets. The liquid feed (Canna; 4 mL of coco A and 4 mL of coco B per 1 L water; electric conductivity EC=2.0 mS/cm) was added after N Bs were generated. pH in all buckets was adjusted to 6.0. EC and pH were checked and adjusted to the right level daily. Plants were grown in hydroponics for 14 days. Plant growth was monitored and compared to controls. Plant growth was determined by measurement of major growth parameters including plant height, whole plant fresh weight and number of stems. T-test in GenStat for Windows 21st Edition (VSN International Ltd., Hemel Hempstead, U.K.) was used to analyse the growth parameters. Those experiments showed that plants treated with the coco A+B and ONB had significantly bigger plant biomass compared to plants treated with coco A+B and tap water (FIG. 12).

### Example 6

Plant growth regulators (PGRs) are chemicals used to modify plant growth. For example, PGRs can be used to increase or stop branching, suppress or stimulate shoot growth, increase flowering or shorten time to flowering, remove excess fruit, alter fruit maturity or block biosynthesis of plant hormones. Numerous factors affect PGRs performance, including how well the chemical is absorbed by the plant. Delivery of PGRs with ONB should improve absorption of PGRs by the plant.

Hydroponic experiments were set up in glasshouse conditions: day temp. 25° C., night temp. 18° C., 16/8 h day/night and 150 μmol m⁻² s⁻¹ light intensity. Cannabis (Cs) apical cuttings were treated with Gibberellic acid A3 (GA3; Duchefa, G0907) at 12 mg/L final concentration, similar to Mansouri et al. (2011) or with DL-carnitine hydrochloride (Merck S7021474 Cas-No 461-05-2, 8.41774.0025) at 1 mM/L final concentration, as in Signem Oney-Birol (2019).

All solutions were prepared first in buckets, pH adjusted to 6.0. The liquid feed (Canna coco A+B) was added at the concentration: 4 mL of coco A and 4 mL of coco B per 1 L water; electric conductivity EC=2.0 mS/cm. Next the solutions were run through a fine bubble generator (Anzaikantetsu Co, AZ-FB-20ASVV) with a 0.75 standard litres per minute (SLPM) O₂ flow and 800 L/H water flow. Each 25 L bucket was run for 30 min through the nozzle. EC and pH were checked and adjusted to the right level daily. Plants were grown in hydroponics for 14 days. Plant growth was monitored and compared to controls. Plant growth was determined by measurement of major growth parameters including plant height, whole plant fresh weight and number of stems. One-way design analysis of variance (ANOVA) and Tukey's 95% confidence intervals test in GenStat for Windows 21st Edition (VSN International Ltd., Hemel Hempstead, U.K.) were used to analyse the growth parameters. The results are shown in FIG. 13 which shows significant increases in growth by using PGRs and ONB under hydroponic conditions.

### Example 7

Seedlings of various lettuce varieties (Lactuca sativa) were exposed to ultrasonic fog generated from water that contained air nanobubbles carrying MVOCs. After 14 days, treated plants showed a significant increase in fresh weight.

Albuterol Sulfate 98.5% (Spectrum Chemical, New Brunswick, NJ, USA) was diluted in tap water at 1.65 mg/L. Five litres was then placed in a container that was pressurized by an air compressor at 1.3 bar. Gas flow from the pressurized container was directed to a nanobubble generator installed in a recirculating flow of water totaling 60 L. After a minimum of 2 hr treatment, 4 L of nanobubble treated water was removed from the recirculating system and placed in a rectangular plastic reservoir with a total capacity of 6 L. A three head ultrasonic fog generator was then placed in the reservoir, and the reservoir was placed in an enclosed plant growth chamber. Seeds of lettuce (Lactuca sativa, var. Tango) were planted in 2.5 cm square cells filled with ProMix growing media. Upon shoot emergence, seedlings were placed in the growth chamber and fog treatments began. A ‘control’ group of plants was placed in a different section of the growth chamber that was not subjected to any treatment. Growing parameters within the chambers were maintained at levels suitable for the crop, including a photoperiod of 16 hr/day. Fog generator operation was controlled by a cycle timer, with an ‘on’ time of 5 min/hr. Fog application was only made during the light period of the day. Plants were irrigated as necessary to maintain proper moisture levels within the cells. Reservoir levels were maintained as necessary by adding treated water from the aforementioned nanobubble recirculating flow system. After 14 days, all plants were harvested and fresh weight recorded. Treated plants showed an average increase in weight of over 30% compared to control plants. The results are shown in FIG. 14a. Similar results were demonstrated in respect of Lactuca sativa var. Iceberg at 22 days, as shown in FIG. 14b.

Each document, reference, patent application or patent cited in this text is expressly incorporated herein in its entirety by reference, which means it should be read and considered by the reader as part of this text. That the document, reference, patent application or patent cited in the text is not repeated in this text is merely for reasons of conciseness.

Reference to cited material or information contained in the text should not be understood as a concession that the material or information was part of the common general knowledge or was known in any country.

Although the invention has been particularly shown and described with reference to particular examples, it will be understood by those skilled in the art that various changes in the form and details may be made therein without departing from the scope of the present invention.

### CAPTIONS TO FIGURES

FIG. 1—Uptake of CY3 labelled DNA oligos in plant tissues from Cannabis sativa (Cs); Nicotiana benthamiana (Nb); Hordeum vulgare (Hv); and Ocimum basilicum (Ob) with and without Oxygen Nanobubbles (ONB)
a) Cs root after 30 hr incubation with CY3 labelled DNA oligo in 0 water (left), water (middle) or ONB water (right).
b) Cs leaf after 30 hr incubation with CY3 labelled DNA oligo in 0 water (left), water (middle) or ONB water (right). 10× magnification.
c) Nb leaf after 24 hr incubation with CY3 labelled DNA oligo in water (left) or ONB water (right). 10× magnification.
d) Hv leaf after 24 hr incubation with CY3 labelled DNA oligo in water (left) or ONB water (right). 10× magnification.
e) Ob leaf after 24 hr incubation with CY3 labelled DNA oligo in water (left) or ONB water (right). 10× magnification.

FIG. 2—Uptake of CY3 labelled PDS oligo in plant tissues from Cannabis sativa (Cs) with and without Oxygen Nanobubbles (ONB)
a) Cs root after 30 hr incubation with CY3 labelled PDS oligo in tap water (left) or ONB water (right).
b) Cs leaf after 3 hr incubation with CY3 labelled PDS oligo in tap water (left) or ONB water (right). 10× magnification.
c) Cs leaf after 30 hr incubation with CY3 labelled PDS oligo in tap water (left) or ONB water (right). 20× magnification.

FIG. 3—Range of plant material (rooted cuttings or seedlings) used for DNA oligo treatment
a) Cannabis sativa (Cs) rooted plants in 50 ml falcon tubes.
b) Cs rooted cutting in Eppendorf.
c) Cs rooted cuttings in coco coir.
d) Nicotiana benthamiana (Nb) seedlings in eppendorfs.
e) Hordeum vulgare seedling in universal tube.
A range of ages from 3-6 weeks old were used.

FIG. 4—Phenotype of Cannabis sativa (Cs), Nicotiana benthamiana (Nb) and Hordeum vulgare (Hv) plants following uptake of PDS antisense oligos with and without Oxygen Nanobubbles (ONB)
a) Cs leaf showing localised phenotype 5 days after incubation with PDS oligos in ONB water.
b) Hv leaves showing PDS phenotype (right) 20 days after incubation with PDS oligos in ONB water. Control without ONB (left).
c) Nb new leaves showing PDS phenotype 20-37 days after incubation with PDS oligos in ONB water.

FIG. 5—PDS mRNA levels in Cannabis sativa (Cs) and Nicotiana benthamiana (Nb) leaves after uptake of PDS antisense oligos with and without Oxygen Nanobubbles (ONB)
a) PDS mRNA levels in Cs leaf 5 days after incubation with PDS antisense oligos in water (left) and ONB water (right) relative to eF1a control gene.
b) PDS mRNA levels in Nb leaf 37 days after incubation with PDS antisense oligos in water (left) and ONB water (right) relative to eF1a control gene.

FIG. 6—Size distribution of nanobubbles measured in ONB water prepared for oligo treatments
Size distribution by intensity of nanobubbles measured in ONB water sample 5 days after collection (dashed) and 12 days after collection (solid).

FIG. 7—The effect of oxygen nanobubbles (ONBs) on Agrobacterium uptake by Nicotiana benthamiana (Nb) seedlings
a) Nb seedlings incubated in MS30 medium+/−Agrobacterium expressing GUS, +/−ONB for two days prior to staining for GUS activity. The control in the middle was treated without Agrobacterium or ONB.
b) Nb seedlings immersed in ¼ MS10 medium containing Agrobacterium expressing GUS with ONB (left) or without (right) for four days prior to staining for GUS activity.

FIG. 8—The effect of oxygen nanobubbles (ONBs) on CRISPR/Cas9 based gene editing efficiency in Nicotiana tabacum (Nt) seedlings
a) CRISPR/Cas9 construct expressing tomato-codon-optimised Cas9 (LeCas9) and a guide RNA (gRNA) to target β-Glucosidase (GUS) gene.
b) target GUS gene is made of two defective partial GUS fragments missing the 5′ or 3′ end. Upon DNA break, homologous recombination (HR) between the two fragments restores the functional GUS gene.
c) These rare spontaneous HR events are detected as blue spots on seedlings (white arrow).
d) More blue spots were detected in the presence of ONBs (right) compared with the control (left).
e) Enlarged leaf areas (AE1-3) of the seedling in the panel e, right and EA4 of another seedling.
f) The total number of blue spots were scored in each treatment:
   Tap_C=tap water and Agrobacterium control
   Tap_CRISPR=tap water and Agrobacterium CRISPR_GUS
   ONBs_C=ONBs and Agrobacterium control
   ONBs_CRISPR=ONBs and Agrobacterium CRISPR_GUS.

FIG. 9—Production of oxygen (or other gas) nanobubble water with volatiles in recirculating water

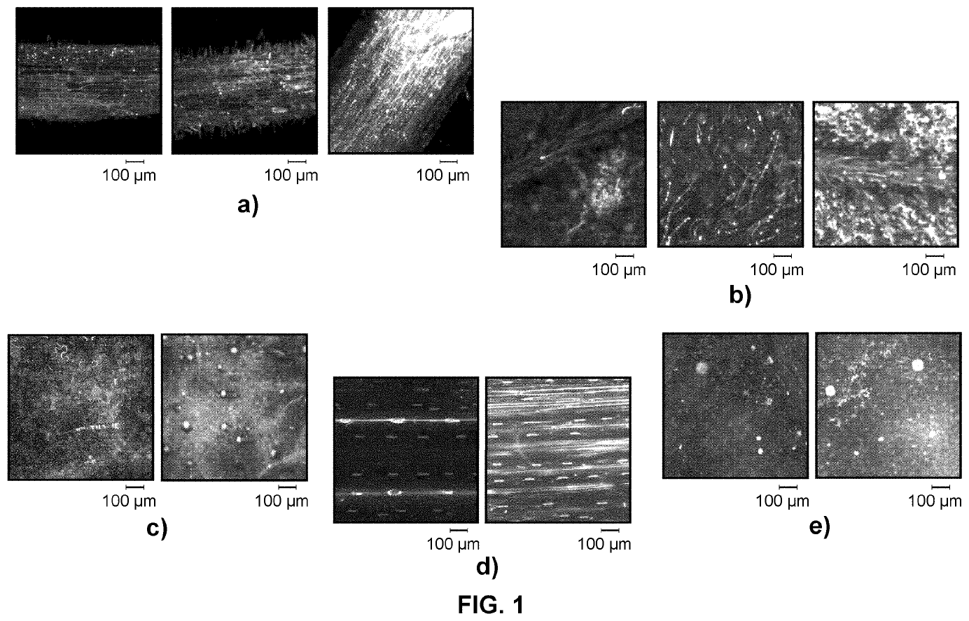

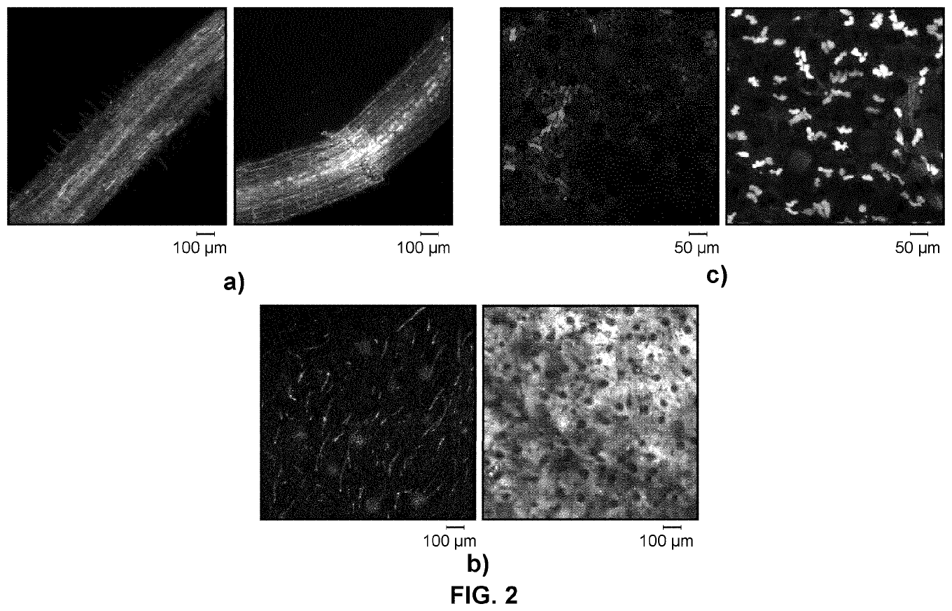

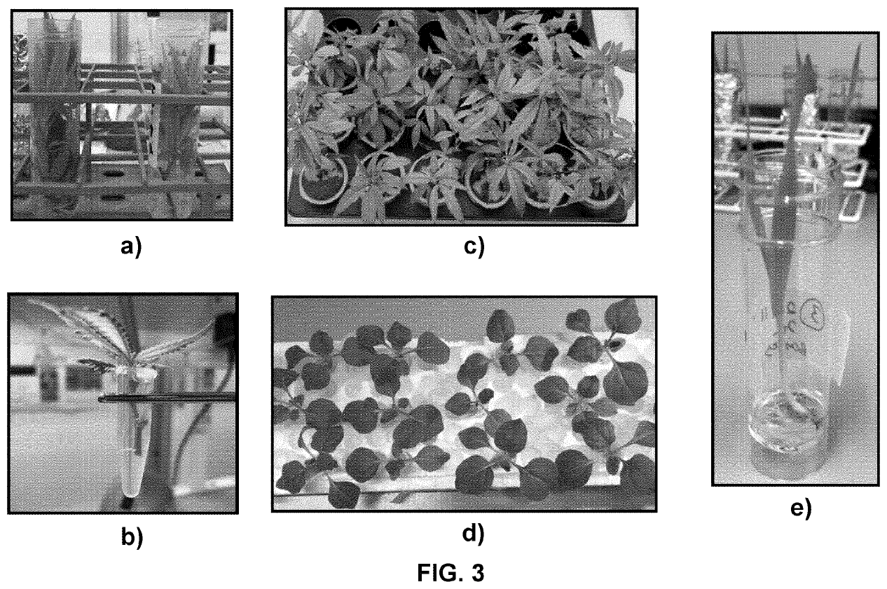

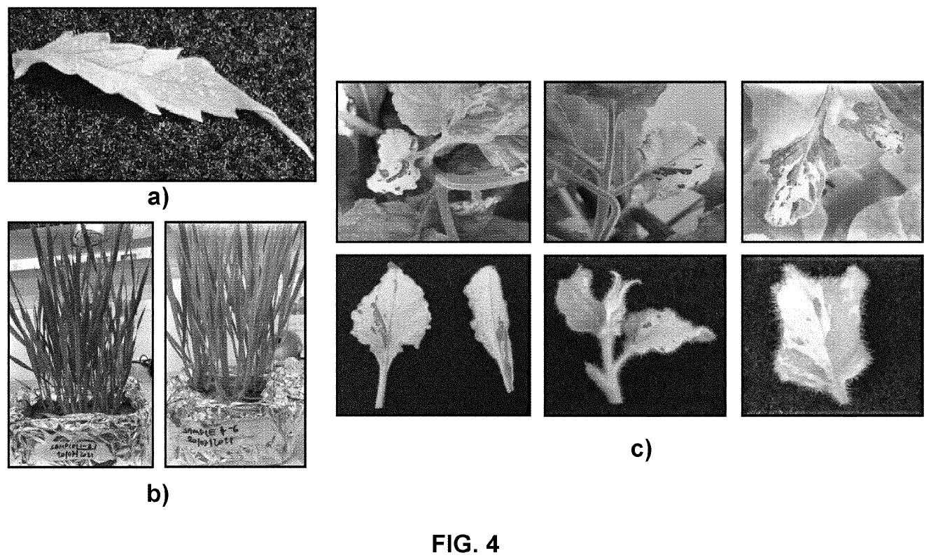

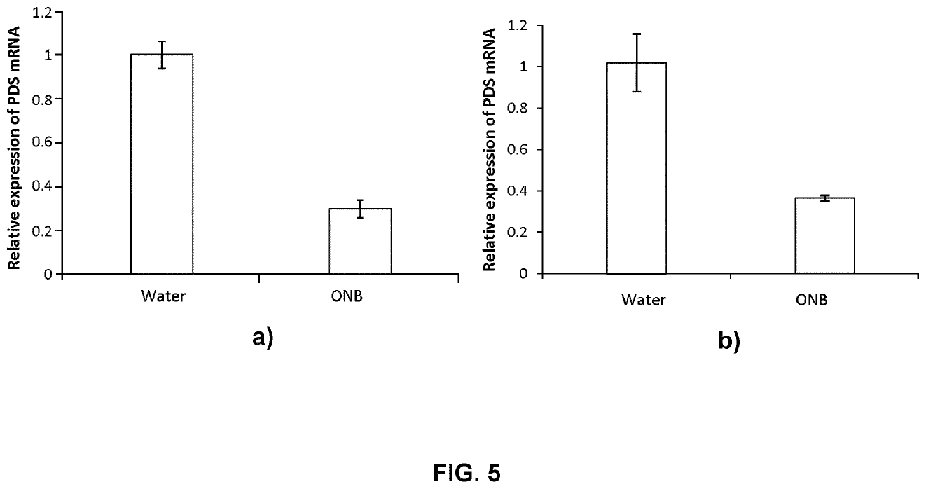

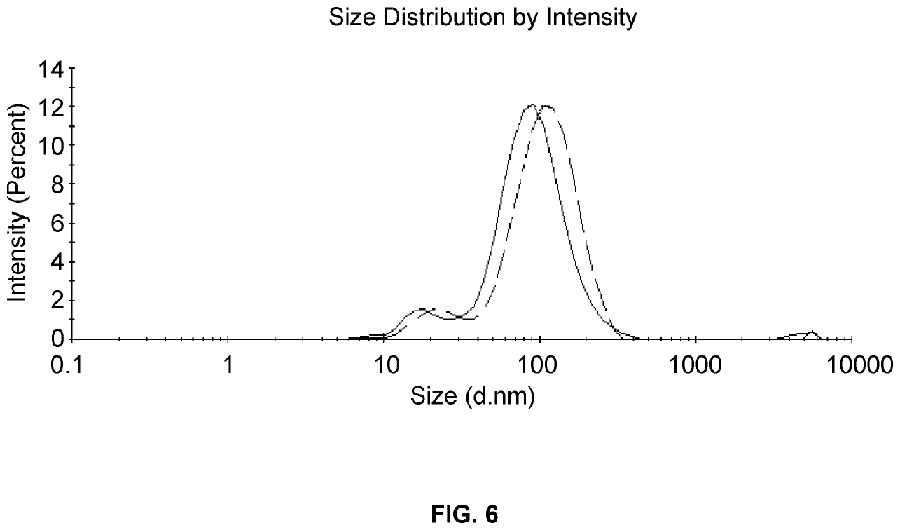

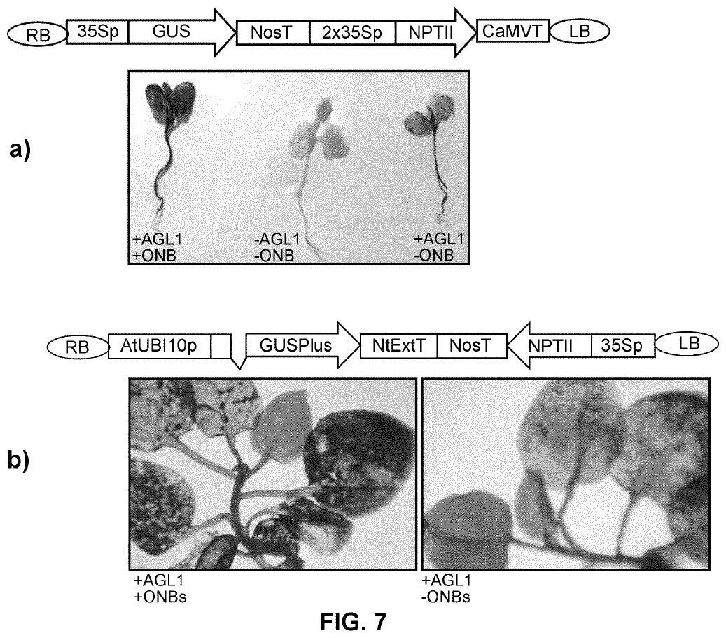

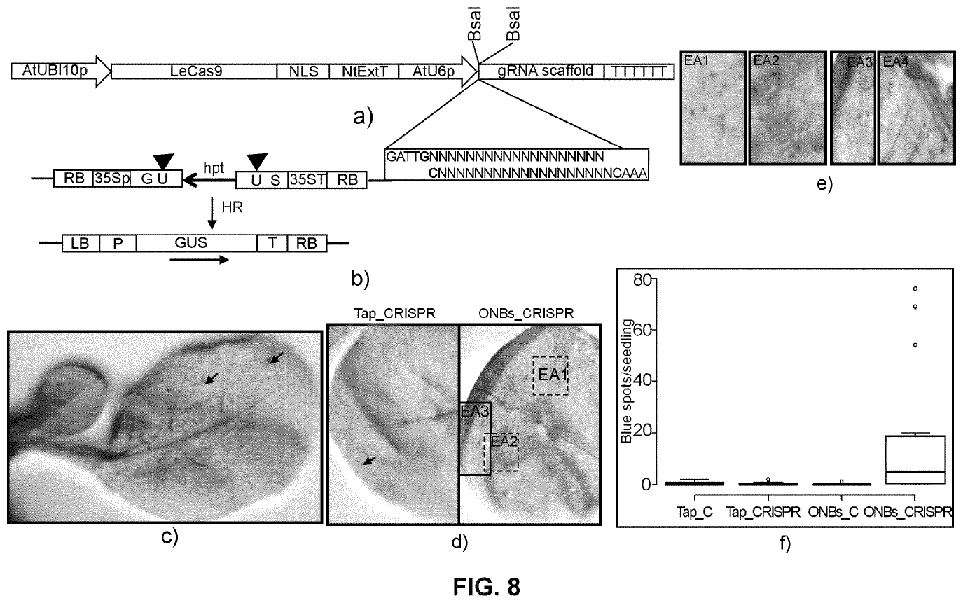

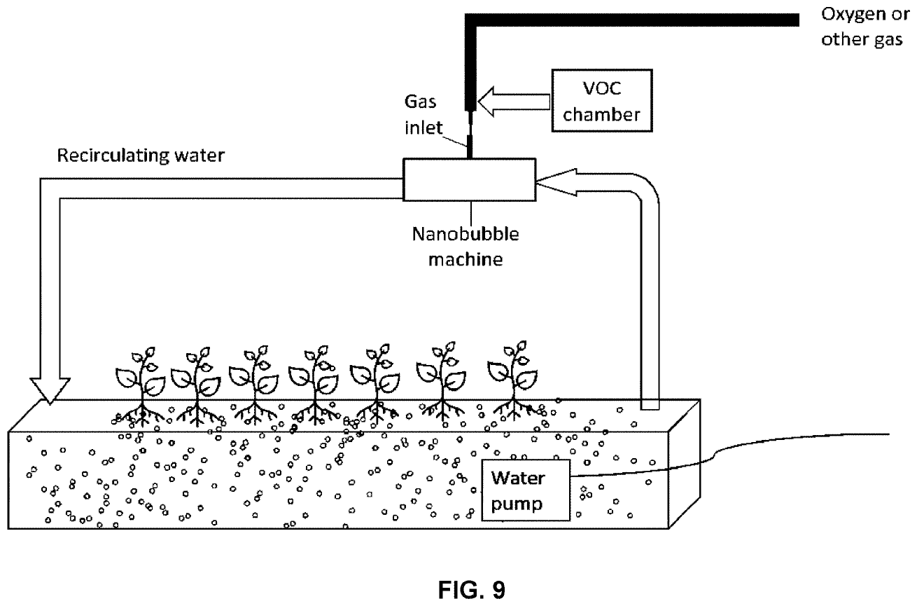

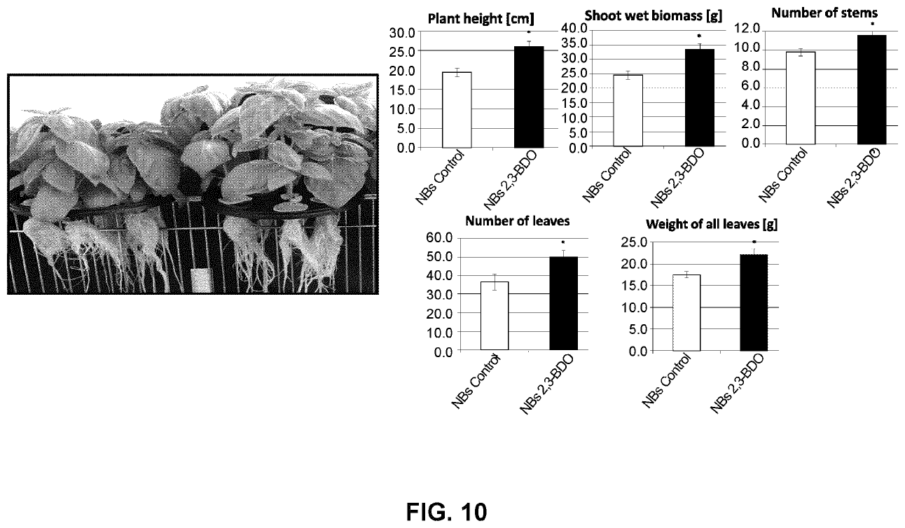

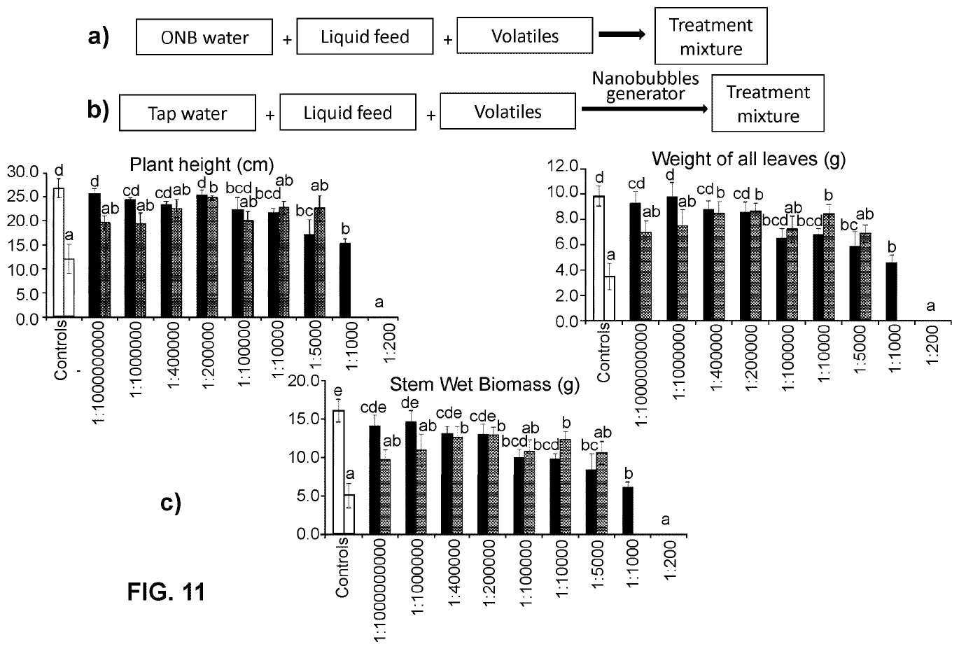

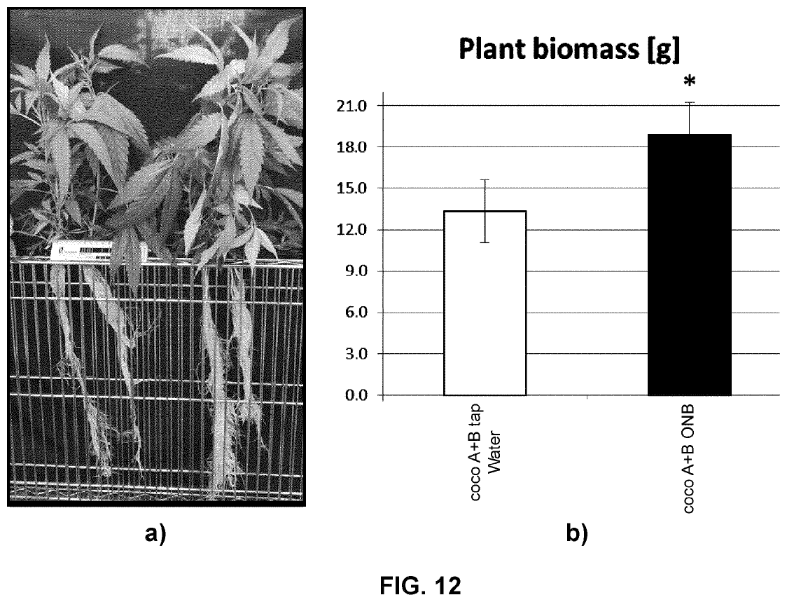

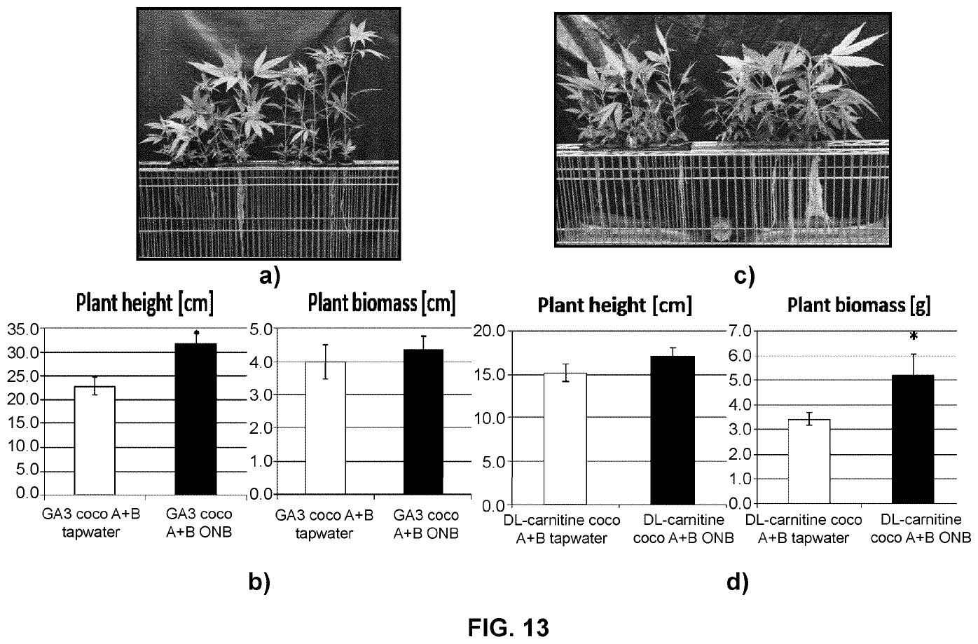

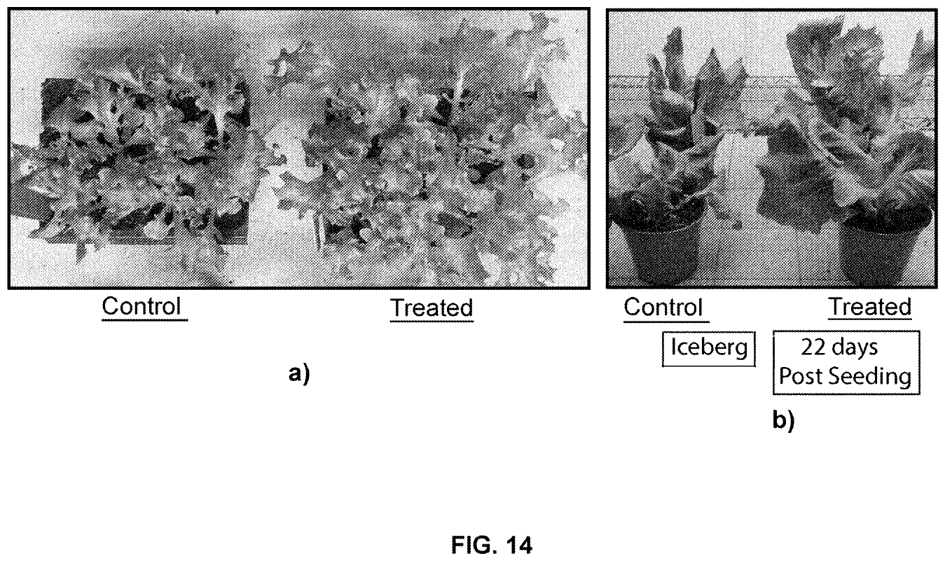

FIG. 10—The use of nanobubbles as a delivery system for volatile compounds to improve growth in Ocimum basilicum seedlings
a) Ocimum basilicum (Ob) seedlings after 21 days growing in recirculating nanobubble water (left) and nanobubble water with a volatile compound.
b) Effect of nanobubbles (grey bars) and nanobubbles with volatile (black) on a number of growth parameters in Ob seedlings after 21 days growing in recirculating hydroponic systems.

FIG. 11—Optimisation of the delivery method of volatiles with nanobubbles (NBs) to plants through the roots in hydroponics with recirculating water. Different concentrations of volatiles and two methods of preparation of volatiles with plant feed and NBs were tested.
a) ONB water was prepared first, then plant liquid feed (in concentration that was optimal for plants growth in the tap water) and different concentration of volatiles were added to the ONB water.
b) Volatiles and liquid feed mixtures were added to tap water and then the mixtures were run through nanobubble generator.
c) The first preparation method (solid grey and black bars) showed the Ocimum basilicum control plants were the highest and had biggest stem biomass; plant growth was inhibited in the highest concentrations of the volatiles. The second NBs mixture preparation method (grey and black pattern bars) showed control plant growth inhibition, dose response to volatile and plant growth improvement of plants treated with volatiles comparing to the control plants.

FIG. 12—The use of nanobubbles as a delivery system for liquid feed to improve growth in Cannabis sativa plants.
a) Cannabis sativa (Cs) cuttings after 14 days growing in liquid feed delivered with recirculating tap water (left) or with recirculating ONB (right).
b) Effect of tap water (grey bar) and ONB (black bar) on uptake of liquid feed on wet plant biomass in Cs cuttings after 14 days growing in recirculating hydroponic system.

FIG. 13—The use of nanobubbles as a delivery system for Plant Growth Regulators (gibberellic acid and DL-carnitine)
a) Uptake of gibberellic acid in Cannabis sativa cuttings delivered with recirculating tap water (left) or with recirculating ONB (right).
b) Effect of tap water (grey bar) and ONB (black bar) on uptake of gibberellic acid in Cannabis sativa cuttings.
c) Uptake of DL-carnitine in Cannabis sativa cuttings delivered with recirculating tap water (left) or with recirculating ONB (right).
d) Effect of tap water (grey bar) and ONB (black bar) on uptake of DL-carnitine in Cannabis sativa cuttings.

FIG. 14—Delivery of volatiles in air nanobubbles as an ultrasonic fog to the leaves for improved growth of Lactuca sativa varieties
a) Lactuca sativa (Tango variety) treated with ultrasonic fog to the leaves containing air nanobubbles with volatile compound (right) compared to control with no fogging (left) after 14 days.
b) Lactuca sativa (Iceberg variety) treated with ultrasonic fog to the leaves containing air nanobubbles with volatile compound (right) compared to control with no fogging (left) 22 days.

1. A plant cultivation system comprising:
(i) a micro- and/or nanobubble generating apparatus for generating micro- and/or nanobubbles from at least one gas;
(ii) a plant application medium comprising a substance, a carrier medium and micro- and/or nanobubbles formed from the at least one gas by the micro- and/or nanobubble generating apparatus; and
(iii) an applicator system to apply the plant application medium comprising the substance to at least one locus of a plant.

2. A system as claimed in claim 1 wherein the applicator system comprises a system for immersion of roots and/or leaves of the plant in the plant application medium.

3. A system as claimed in claim 1 wherein the applicator system comprises a system for spraying, fogging or misting the plant with the plant application medium, optionally wherein the at least one gas comprises carbon dioxide and the applicator system comprises a system for misting leaves of the plant.

4. A system as claimed in claim 1 wherein the applicator system is in fluid communication with the micro- and/or nanobubble generating apparatus.

5. A system as claimed in claim 1 comprising a hydroponic plant cultivation system.

6. A system as claimed in claim 1 wherein the micro- and/or nanobubble generating apparatus is a nanobubble-generating apparatus.

7. A system as claimed in claim 1 wherein the substance is or includes at least one compound, vector or nanomaterial, or an epigenetic regulator.

8. A system as claimed in claim 1 wherein the substance is or includes at least one substance selected from: volatile organic compounds (VOCs); transgenes, nucleic acids, DNAs, RNAs, siRNA, antisense oligonucleotides, synthetic or native DNA or RNA, synthetic or native DNA or RNA up to 500 nucleotides; plant growth regulators, gibberellins, auxins, abscisic acid, cytokinins and ethylene; epigenetic regulators; RNAi vectors, expression vectors, viral vectors, mono-polysaccharides; polyphenols; terpenoids; proteins or peptides, peptides up to 150 amino acids, up to 50 amino acids; nanomaterials, a nanomaterial selected from: lipid nanoparticles, carbon nanotubes, copper nanoparticles, iron or iron oxide nanoparticles, manganese or manganese oxide nanoparticles, titanium dioxide nanoparticles, and zinc or zinc oxide nanoparticles; and plant protection products.

9. A system as claimed in claim 8 wherein the substance is or includes at least one substance selected from VOCs, RNAs, siRNA, antisense oligonucleotides, epigenetic regulators, peptides, RNAi, expression and viral vectors.

10. A process for delivering a substance to cells of a plant, the process comprising:
(i) providing a plant application medium comprising a substance, a carrier medium and micro- and/or nanobubbles of at least one gas; and
(ii) applying the plant application medium to a locus of a plant.

11. A process as claimed in claim 10 wherein the step of applying the plant application medium to the plant comprises applying the plant application medium to roots and/or leaves of the plant, by immersion, spraying, fogging or misting.

12. A process as claimed in claim 10 wherein the substance and micro- and/or nanobubbles are transported or translocated from the locus of the plant to at least one plant cell, wherein the substance and micro- and/or nanobubbles are transported or translocated from a first plant tissue to a second plant tissue.

13. A process as claimed in claim 10 wherein the substance is or includes at least one compound, vector or nanomaterial, wherein the substance is or includes at least one substance selected from: volatile organic compounds (VOCs); transgenes, nucleic acids, DNAs, RNAs, siRNA, antisense oligonucleotides, synthetic or native DNA or RNA, synthetic or native DNA or RNA up to 500 nucleotides; plant growth regulators, gibberellins, auxins, abscisic acid, cytokinins and ethylene; epigenetic regulators; RNAi vectors, expression vectors, viral vectors, mono-polysaccharides; polyphenols; terpenoids; proteins or peptides, peptides up to 150 amino acids, up to 50 amino acids; nanomaterials, a nanomaterial selected from: lipid nanoparticles, carbon nanotubes, copper nanoparticles, iron or iron oxide nanoparticles, manganese or manganese oxide nanoparticles, titanium dioxide nanoparticles, and zinc or zinc oxide nanoparticles; and plant protection products.

14. A process as claimed in claim 13 wherein the substance is or includes at least one substance selected from VOCs, RNAs, siRNA, antisense oligonucleotides, epigenetic regulators, peptides, RNAi, expression and viral vectors, wherein the substance includes an epigenetic regulator.

15. A plant application medium, for applying to a locus of a plant, the medium comprising a substance, a carrier medium and micro- and/or nanobubbles of at least one gas.

16. A medium as claimed in claim 15 wherein the substance is or includes at least one compound, vector or nanomaterial, wherein the substance is or includes at least one substance selected from: volatile organic compounds (VOCs); transgenes, nucleic acids, DNAs, RNAs, siRNA, antisense oligonucleotides, synthetic or native DNA or RNA, synthetic or native DNA or RNA up to 500 nucleotides; plant growth regulators, gibberellins, auxins, abscisic acid, cytokinins and ethylene; epigenetic regulators; RNAi vectors, expression vectors, viral vectors, mono-polysaccharides; polyphenols; terpenoids; proteins or peptides, peptides up to 150 amino acids, up to 50 amino acids; nanomaterials, a nanomaterial selected from: lipid nanoparticles, carbon nanotubes, copper nanoparticles, iron or iron oxide nanoparticles, manganese or manganese oxide nanoparticles, titanium dioxide nanoparticles, and zinc or zinc oxide nanoparticles; and plant protection products.

17. A medium as claimed in claim 16 wherein the substance is or includes at least one substance selected from VOCs, RNAs, siRNA, antisense oligonucleotides, epigenetic regulators, peptides, RNAi, expression and viral vectors, wherein the substance includes an epigenetic regulator.

18. A plant to which a medium as claimed in claim 15 has been applied to a locus thereof, wherein the locus is roots of the plant or leaves of the plant.

19. A process for inducing a change in a phenotype, chemistry or physiology of a plant by delivering an epigenetic regulator to a plant, the process comprising:
(i) providing a plant application medium comprising a substance, a carrier medium and micro- and/or nanobubbles of at least one gas; and
(ii) applying the plant application medium to a plant, whereby the epigenetic regulator enters at least one plant tissue of the plant and a subsequent change is induced in the phenotype, chemistry or physiology of the plant.

20. A process according to claim 19 wherein the epigenetic regulator is at least one epigenetic regulator selected from: volatile organic compound(s) (VOC(s)), fungal, microbial or plant VOCs; RNA, siRNA; antisense oligonucleotides; peptides; RNAi vectors; expression vectors; viral vectors; and plant growth regulators.

21. A process according to claim 19 wherein, in use of the process, the epigenetic regulator induces DNA methylation, RNA methylation, histone methylation or histone acetylation, in one or more flowering loci.

22. A process according to claim 19 wherein the plant epigenetic regulator is or includes a nucleic acid.

23. A process for editing a gene of a plant, the process comprising:
(i) providing a plant application medium comprising a gene editing substance, a carrier medium and micro- and/or nanobubbles of at least one gas; and
(ii) applying the plant application medium to a plant, whereby the substance enters at least one plant cell.

24. A process according to claim 23 wherein the substance comprises a CRISPR/Cas9 construct, wherein the substance comprises a CRISPR/Cas9 construct introduced by an Agrobacterium.

25. A process for delivering a plant or crop protection product into a plant, the process comprising:
(i) providing a plant application medium comprising a substance, a carrier medium and micro- and/or nanobubbles of at least one gas; and
(ii) applying the plant application medium to a plant;
wherein the substance is or includes at least one plant or crop protection product, comprising a herbicide, pesticide, an insecticide, nematocide, or acaricide;
wherein, in use of the process, the plant or crop protection product is absorbed into a plant tissue, a leaf or root tissue.

26. A process for delivering an antisense oligonucleotide to a plant, the process comprising:
(i) providing a plant application medium comprising a substance, a carrier medium and micro- and/or nanobubbles of at least one gas; and
(ii) applying the plant application medium to a plant;
wherein the substance is or includes at least one antisense oligonucleotide;
wherein, in use of the process, the antisense oligonucleotide enters at least one plant cell of the plant.

27. A process according to claim 26 wherein the antisense oligonucleotide plant application medium is applied to a root of the plant, wherein the antisense oligonucleotide is translocated from the root of the plant to a leaf of the plant, in use of the process.

28. A system as claimed in claim 1 wherein at least 50% of the micro and/or nanobubbles generated have a diameter of less than about 1000 nm.

29. A system as claimed in claim 1 wherein 100% or about 100% of the micro- and/or nanobubbles generated have a diameter of less than about 1000 nm.

30. A system as claimed in claim 1 wherein the at least one gas is at least one gas selected from oxygen, nitrogen, carbon dioxide and air.

31. A system as claimed in claim 1 wherein the nanobubbles are generated using an electric field.

32. A system as claimed in claim 1 wherein the nanobubbles generated maintain stability for about 2 years or longer.

33. A process as claimed in claim 10 further comprising a pre-treatment step wherein rooted shoots of the plant are incubated in an oxygen nanobubble water for one to two days prior to application of the medium.

34. A process as claimed in claim 10 wherein a mixture of a nanobubble water and one or more substance to alter gene expression is provided to a plant at any time in the life cycle of the plant to induce one or more epigenetic changes in real time.

35. A system as claimed in claim 1, wherein the plant is Cannabis sativa, Nicotiana benthamiana, Hordeum vulgare, Nicotiana tabacum. Lactuca sativa or Ocimum basilicum.
